(19) United States
(12) Patent Application Publication
(10) Pub. No.: US 2024/0368225 A1
(43) Pub. Date: Nov. 7, 2024

(54) SELECTIVE SMALL MOLECULE AGONISTS AND PARTIAL AGONISTS OF TRK RECEPTORS

(71) Applicant: THE TEXAS A&M UNIVERSITY SYSTEM, College Station, TX (US)

(72) Inventor: Kevin BURGESS, College Station, TX (US)

(21) Appl. No.: 18/566,143

(22) PCT Filed: Jun. 1, 2022
(86) PCT No.: PCT/US2022/031754

§ 371 Date: Dec. 1, 2023

Related U.S. Application Data
(60) Provisional application No. 63/195,868, filed on Jun. 2, 2021.
(60) Provisional application No. 63/270,266, filed on Oct. 21, 2021.
(60) Provisional application No. 63/337,335, filed on May 2, 2022.

(51) Int. Cl. C07K 7/54; A61K 38/00; C07K 5/062; C07K 5/072; C07K 5/083; C07K 5/103

(52) U.S. Cl. CPC C07K 7/54; C07K 5/06026; C07K 5/06095; C07K 5/0808; C07K 5/081; C07K 5/101; A61K 38/00

(57) ABSTRACT

The present disclosure relates to macrocyclic compounds, pharmaceutical compositions containing macrocyclic compounds, and methods of using macrocyclic compounds to treat disease, such as diseases of the eye.

### CROSS-REFERENCE TO RELATED APPLICATIONS

This application claims priority under 35 U.S.C. § 119(e) to U.S. Provisional Application Ser. No. 63/195,868 filed on Jun. 2, 2021, to U.S. Provisional Application Ser. No. 63/270,266 filed on Oct. 21, 2021, and to U.S. Provisional Application Ser. No. 63/337,335 filed on May 2, 2022, the entire disclosures of which are incorporated herein by reference.

### TECHNICAL FIELD

The present disclosure relates to macrocyclic compounds, pharmaceutical compositions containing macrocyclic compounds, and methods of using macrocyclic compounds to treat disease, such as diseases of the eye.

### BACKGROUND

Vision depends on corneal clarity. Cornea innervation is provided by the trigeminal nerve; stromal nerves enter the cornea through the stroma then are found perpendicular to the epithelium and projecting into the intraepithelial corneal nerves. Most corneal nerves are sensory, but some sympathetic and parasympathetic ones provide blink reflexes. The cornea is one of the most densely innervated organs in the body, and the corneal epithelium supports nerves by producing neurotrophic factors. Diseases that affect either the epithelium (eg dry eye) or trigeminal branch (herpes-simplex, -zoster or surgery) can impact corneal nerves and lead to epithelium and nerve alterations, as in neurotrophic keratitis (NK). Herpes zoster corneal infection is the most common cause of NK, and dry eye is the second.

NK occurs at a rate of >5/10,000 (ie currently ˜164,000 cases in the US); it can lead to blindness via corneal melting and perforation. Clinically, NK is characterized by loss of cornea sensitivity, epithelium breakdown and poor healing. The three stages of NK have been defined based on stroma involvement. Here we refer to mild NK (stage 1) and severe NK (stage 2-3, neurotrophic ulcers when there is an epithelial defect, and stroma involvement). Unfortunately, NK can be resistant to clinical treatment. Further, NK is not a prime target for pharmaceutical companies because its incidence is insufficient to make its treatment extremely profitable. NIH-supported research in this area fills a need that might not otherwise be addressed.

Cenegermin, recombinant nerve growth factor (NGF), is the only approved (FDA and European agencies) drug for NK treatment. It emerged because research showed that NGF can promote healing of corneal ulcers, NGF levels increase in wounded cornea in a rat model, and corneal healing can be stimulated by topical administration of NGF in rabbit and dog models. It has been postulated that these healing roles seem to be related to NGF increasing proliferation of corneal epithelial cells, just as in skin it stimulates growth around wound margins. Furthermore, treatment with NGF increases goblet cell density and production of mucin Muc5ac.

Cenegermin is not an ideal therapeutic. It is expensive because recombinant NGF is difficult to make reproducibly, has a limited shelf life, and requires frequent (>6 times/d), prolonged (>2 months) administration. Moreover, NGF also interacts with another receptor, p75 and the NGF•p75 interaction generally induces apoptosis (FIG. 1). Of special concern here is that p75 overexpression is associated with apoptosis of retinal ganglion cells, 10 and microglia-derived NGF causes cell death in developing retina. Consequently, detrimental side-effects associated with long term use of NGF (Cenegermin) are possible. All these factors point to an urgent unmet need for improved NK therapies.

Neurotrophins are high homologous cytokines that bind the tyrosine kinase receptors, Trk. Crystallographic data for NGF•TrkA is limited due to the usual difficulties crystallizing cell surface receptors, especially complexed with their ligands. However, evidence from the partial structures that have been reported suggest NGF binds via loop regions contacting the transmembrane region. See FIG. 2. Further evidence that the three neurotrophin loops form hot-spots for interaction with Trk receptors come from site directed mutagenesis studies, and preparation of chimeric proteins. Thus, in previous work small loop analogs of NGF were developed, one compound in particular, Tavilermide (D3), because it proved to be a partial agonist of TrkA.

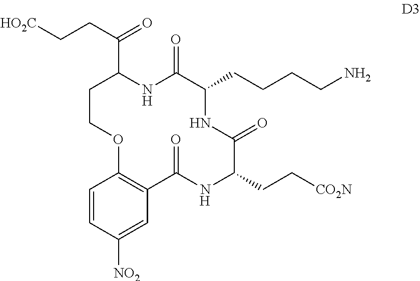

Goblet cells secrete mucins and immunoregulatory factors essential to healthy ocular surfaces, and D3 increased glycoconjugated mucin secretion in vitro. Consequently, tavilermide, D3, entered phase 3 US clinical trials for treatment of dry eye disease. In the original clinical trial for D3, there may have been a problem which prevented publication of trial results, and Mimetogen announced in the Spring of 2021 that D3 has re-entered phase 3 trials. Approval of D3 for dry eye disease may be complicated by the fact that it contains an aromatic nitro group (as discussed below); this functionality is rare in pharmaceuticals because of potential toxicity issues. Alternatively, selectivity of D3 amongst the Trk receptors could conceivably be an issue, but there is no evidence that activation of TrkB or C is detrimental. On the other hand, a compelling feature of D3 is that it does not activate p75. D3 was designed to mimic i+1, i+2 (residues 94,95) of a turn in NGF. See FIGS. 3a-3b.

TrkA agonists stimulate various cell types in the eye, including those that may generate mucins on the cornea. Consequently as noted above, D3 has been investigated as a therapy for dry-eye disease. D3 has the following potential liabilities as a pharmaceutical: (i) it contains a nitro group, and such functionalities can be toxic; (ii) D3 is only a partial agonist, meaning it synergizes with endogenous NGF, but if it has significantly less activity in the absence of that cytokine; and, (iii) it is unclear that it is a sufficiently potent TrkA partial agonist to induce the desired therapeutic effect. Also, D3 is a partial TrkA agonist, and does not bind TrkC.

Therefore, there is an upmet need to develop new compounds that can bind to one or more of TrkA, TrkB, or TrkC. and particular interest is in compounds that can bind to TrkB or TrkC. And in particular, there is an urgent need to develop adnovel macrocyclic mimetics od the NGF loop regions to overcome the defiencies of known compounds, e.g. D3, such as it is potentially valuable to devise alternative NGF loop mimics which do not contain NO₂ functionality, and have enhanced cellular responses.

### SUMMARY

In one aspect, the disclosure relates to strategies to increase cellular potencies of compounds that bind to Trks, such as TrkA, TrkB, and TrkC. In some embodiments, one such strategy to increase cellular potency is to maximize target binding by preparing compounds encapsulating more residues into the NGF loop mimics. It has been discovered that the compounds described herein possess affinity for Trks, such as TrkA, TrkB, and TrkC. In some embodiments, the disclosure provides compounds that are both slightly larger than D3 and fluorescent. It has been discovered that the compounds described herein are full agonists of Trks, such as TrkA, TrkB, and TrkC, in contrast to D3 which is only a partial agonist of TrkA and posseses no TrkC agonism.

In one aspect, the disclosure provides a compound of the formula I

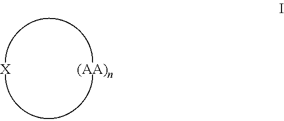

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n);
X is a divalent linking group comprising one or more of a heterocycloalkylene portion, a cycloalkylene portion, a divalent triazole portion, or a divalent dye portion; and
n is 3, 4, 5, 6, 7, or 8.

In one aspect, the disclosure provides a compound of the formula I

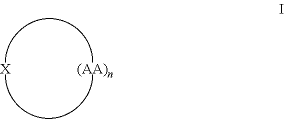

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n);
X is a divalent linking group comprising one or more of a heterocycloalkylene portion, a cycloalkylene portion, a divalent triazole portion, or a divalent dye portion; and
n is 3, 4, 5, 6, 7, or 8.

In one aspect, the disclosure provides a compound of the formula II

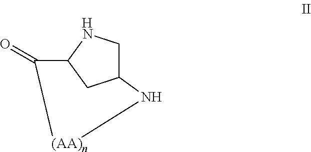

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n); and
n is 3, 4, 5, 6, 7, or 8.

In one aspect, the disclosure provides a compound of the formula II

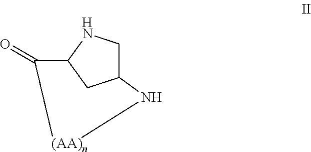

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n); and
n is 3, 4, 5, 6, 7, or 8.

In one aspect, the disclosure provides a compound of the formula III

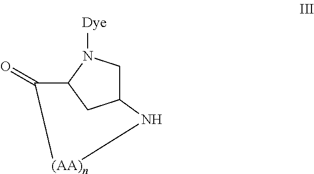

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n);
Dye is a fluorescent dye molecule; and
n is 3, 4, 5, 6, 7, or 8.

In one aspect, the disclosure provides a compound of the formula III

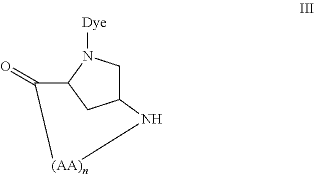

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n);
Dye is a fluorescent dye molecule; and
n is 3, 4, 5, 6, 7, or 8.

In one aspect, the disclosure provides a compound of the formula IV

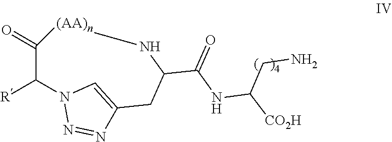

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n);
R′ is a side chain of the amino acid T, V, M, I, K, or S; and
n is 3, 4, 5, 6, 7, or 8.

In one aspect, the disclosure provides a compound of the formula IV

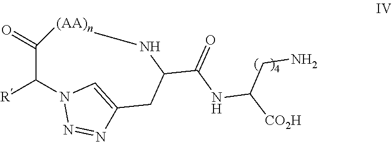

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n);
R′ is a side chain of the amino acid T, V, M, I, K, or S; and
n is 3, 4, 5, 6, 7, or 8.

In one aspect, the disclosure provides a compound of the formula V

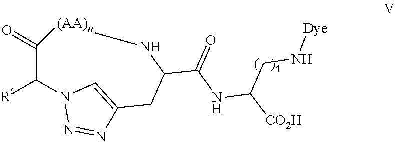

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n);
R′ is a side chain of the amino acid T, V, M, I, K, or S;
Dye is a fluorescent dye molecule; and
n is 3, 4, 5, 6, 7, or 8.

In one aspect, the disclosure provides a compound of the formula V

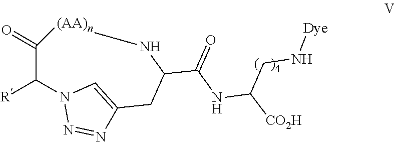

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n);
R′ is a side chain of the amino acid T, V, M, I, K, or S;
Dye is a fluorescent dye molecule; and
n is 3, 4, 5, 6, 7, or 8.

In one aspect, the disclosure provides a compound of the formula XII

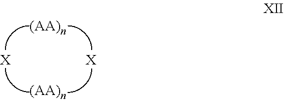

wherein
each (AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n);
each X is a divalent linking group comprising one or more of a heterocycloalkylene portion, a cycloalkylene portion, or a divalent triazole portion; and
n is 3, 4, 5, 6, 7, or 8.

In one aspect, the disclosure provides a compound of the formula XII

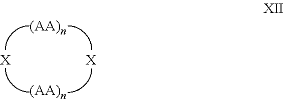

wherein
each (AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n), and the sequences of (AA)ₙ are the same;
each X is a divalent linking group comprising one or more of a heterocycloalkylene portion, a cycloalkylene portion, or a divalent triazole portion; and
n is 3, 4, 5, 6, 7, or 8.

In certain embodiments of the above aspects, the compound of Formula (I)-(XII) is a compound selected from those species described or exemplified in the detailed description below.

In further aspects, the disclosure relates to a pharmaceutical composition comprising at least one compound of Formula (I)-(XII) or a pharmaceutically acceptable salt thereof. Pharmaceutical compositions according to the disclosure may further comprise a pharmaceutically acceptable excipient, carrier, or diluent.

In further aspects, the disclosure relates a compound of Formula (I)-(XII), or a pharmaceutically acceptable salt thereof, for use as a medicament.

In further aspects, the disclosure relates to a method of treating disease in which cell survival is mediated by one or more of TrkA, TrkB, or TrkC, such as an eye disease or neurological disease comprising administering to a subject in need of such treatment an effective amount of at least one compound of Formula (I)-(XII), or a pharmaceutically acceptable salt thereof.

In further aspects, the disclosure relates to use of a compound of Formula (I)-(XII), or a pharmaceutically acceptable salt thereof, in the preparation of a medicament for the treatment of disease in which cell survival is mediated by one or more of TrkA, TrkB, or TrkC, such as an eye disease or neurological disease, and the use of such compounds and salts for treatment of such diseases.

In further aspects, the disclosure relates to a method of stimulating a Trk, such as TrkA, TrkB, or TrkC, comprising contacting a cell comprising one or more of Trk with an effective amount of at least one compound of Formula (I)-(XII), or a pharmaceutically acceptable salt thereof, and/or with at least one pharmaceutical composition of the disclosure, wherein the contacting is in vitro, ex vivo, or in vivo.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A compound of the formula

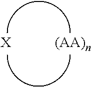

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n);
X is a linking group comprising one or more of a heterocycloalkylene portion, a cycloalkylene portion, a divalent triazole portion, or a divalent dye portion; and
n is 3, 4, 5, 6, 7, or 8.

2. The compound of clause 1, wherein one or more amino acids in the amino acid sequence is a naturally occurring amino acid selected from the group consisting of L-histidine (H), L-threonine (T), L-glycine (G), L-proline (P), L-alanine (A), L-valine (V), L-isoleucine (I), L-leucine (L), L-methionine (M), L-phenylalanine (F), L-tyrosine (Y), and L-tryptophan (W).

3. The compound of clause 1 or 2, wherein one or more amino acids in the amino acid sequence is a naturally occurring amino acid in the D-configuration selected from the group consisting of D-histidine (h), D-threonine (t), D-glycine (g), D-proline (p), D-alanine (a), D-valine (v), D-isoleucine (i), D-leucine (1), D-methionine (m), D-phenylalanine (f), D-tyrosine (y), and D-tryptophan (w).

4. The compound of any one of clauses 1 to 3, wherein one or more amino acids in the amino acid sequence is an unnatural amino acid selected from the group consisting of selenocysteine, citrulline (Cit), hydroxyproline (Hyp), norleucine (Nle), ornithine (Orn), naphtylalanine (Nal), methionine sulfoxide, methionine sulfone, beta-alanine, α-aminobutyric acid, γ-aminobutyric acid, diaminobutyric acid, δ-aminolevulinic acid, 4-amino-benzoic acid, hydroxyproline, and carboxyglutamic acid.

5. The compound of any one of the preceding clauses, wherein the amino acid sequence comprises a mimetic of loop-1, loop-2, or loop-3 of a neurotrophin.

6. The compound of any one of the preceding clauses, wherein the neurotrophin is nerve growth factor, brain-derived neurotrophic factor, neurotrophin-3, or neurotrophin-4.

7. The compound of any one of the preceding clauses, wherein the compound is capable of binding to one or more of TrkA, TrkB, or TrkC.

8. The compound of any one of the preceding clauses, wherein at least two amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine.

9. The compound of any one of the preceding clauses, wherein at least three amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine.

10. The compound of any one of the preceding clauses, wherein at least four amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine.

11. The compound of any one of the preceding clauses, wherein at least five amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine.

12. The compound of any one of the preceding clauses, wherein at least six amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine.

13. The compound of any one of clauses 1 to 9, wherein n is 3.

14. The compound of any one of clauses 1 to 10, wherein n is 4.

15. The compound of any one of clauses 1 to 11, wherein n is 5.

16. The compound of any one of clauses 1 to 12, wherein n is 6.

18. The compound of any one of clauses 1 to 12, wherein n is 7.

18. The compound of any one of clauses 1 to 12, wherein n is 8.

19. The compound of any one of the preceding clauses, wherein the amino acid sequence comprises a sequence selected from the group consisting of -INS-, -snv-, -Vsn-, -DSK-, -SKk-, -sKk-, -Kks-, -ENK-, -nKV-, -vKN-, -Nne-, -ENn-, -DIKG-, -INNS-, -DGKQ-, -DEKQ-, -DMSG-, -VSKG-, -DSKK-, -DIRG-, -TQNS-, -TGNS-, -ENNK-, -DIKGK-, -NINNSVF-, -DGKQA-, -DEKQA-, -DMSGG-, -SKGQ-, -DSKKR-, -DIRGH-, -TQNSP-, -KTQNSPV-, -TQNSG-, -TGNSP-, and -ENNKLV-.

20. The compound of any one of the preceding clauses, wherein X comprises a heterocycloalkylene portion.

21. The compound of clause 20, wherein X is of the formula

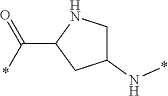 [SMILES: *NC1CNC(C(*)=O)C1]

wherein each * represents a point of covalent attachment to the rest of the compound.

22. The compound of clause 21, wherein X further comprises a dye molecule covalently attached thereto.

23. The compound of clause 22, wherein X has the formula

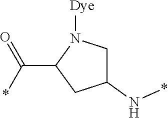

wherein each * represents a point of covalent attachment to the rest of the compound, and dye is a fluorescent dye molecule.

24. The compound of clause 23, wherein the fluorescent dye molecule is a fluorescein dye or dansyl dye.

25. The compound of any one of clauses 1 to 19, wherein X comprises a divalent triazole portion.

26. The compound of clause 25, wherein X comprises a divalent triazole of the formula

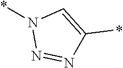 [SMILES: *c1cn(*)nn1]

wherein each * represents a point of covalent attachment to the rest of the compound.

27. The compound of clause 26, wherein X comprises the structure

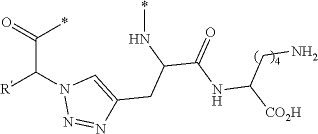

wherein each * represents a point of covalent attachment to the rest of the compound, and R′ is a side chain of the amino acid T, V, M, I, K, or S.

28. The compound of clause 27, wherein X further comprises a dye molecule covalently attached thereto.

29. The compound of clause 28, wherein X has the formula

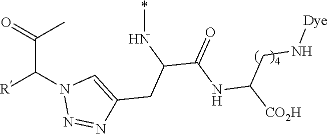

wherein each * represents a point of covalent attachment to the rest of the compound, R′ is a side chain of the amino acid T, V, M, I, K, or S, and dye is a fluorescent dye molecule.

30. The compound of clause 29, wherein the fluorescent dye molecule is a fluorescein dye or dansyl dye.

31. The compound of any one of clauses 1 to 18, wherein the amino acid sequence comprises a sequence selected from the group consisting of -CDEKQC-, -CINNSC-, -CDIKGC-, -CDGKQC-, -CENNKC-, -CDIRGC-, -CTGNSC-, -CTQNSC-, -CDMSGC-, -CVSKGC-, -CDSKKC-, -CDLRGC-, -CAGGSC-, -CDAQGC-, and -CDIKGC-, and the cysteine residues in the sequence are covalently attached to X via the cysteine sulfur atom.

32. The compound of clause 31, wherein the -OH in the carboxylic acid groups of the cysteine residues in sequence are replaced by amide or a protected amide groups.

33. The compound of any one of clauses 1 to 18, 31 or 32, wherein X comprises a divalent dye portion.

34. The compound of clause 33, wherein the divalent dye portion is a fluorescent dye.

35. The compound of clause 34, wherein the divalent dye portion is a bodipy dye.

36. The compound of clause 35, wherein the divalent dye portion is of the formula

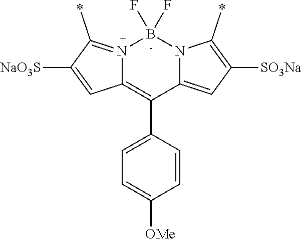

wherein each * represents a point of covalent attachment to the sulfur atom in a cysteine residue in the sequence.

37. A pharmaceutical composition comprising a compound of any one of clauses 1 to 36, and optionally one or more excipients, carriers, or diluents.

38. A method of treating disease mediated by one or more of TrkA, TrkB, or TrkC, comprising administering to a subject a compound of any one of clauses 1 to 36, or the pharmaceutical composition of clause 34.

39. The method of clause 38, wherein the disease is glaucoma, dry eye disease, retinitis pigmentosa, or neurotrophic keratitis.

### BRIEF DESCRIPTION OF THE DRAWINGS

FIG. 1 is a carton showing that small molecules that selectively activate Trk receptors may be useful in treating disease by avoiding effects induced by activation of p75.

FIG. 2 is a carton showing the loop regions of NGF that have been proposed as the binding domain of NGF to the transmembrane region of TrkA.

FIGS. 3A-3B are cartoons showing the loop regions of NGF. FIG. 3A is a catoon showing the residues DEKQ in loop 4, a β-turn in murine NGF. FIG. 3B is a cartoon showing a ribbon diagram of the amino acid sequence for NGF, with loop regions highlighted by residue numbers (27-32, 42-49, and 93-96).

FIG. 4 is a graph of cell survival in HeLa-TrkA cells for each compound (50 μM) in Series 1. The right bar shows cell survival after treatment with suboptimal (0.2 nM) levels of NGF and the left bar shows cell survival after no treatment of NGF.

FIG. 5 is a graph of cell survival in HeLa-TrkA cells for each compound (50 μM) in Series 2. The right bar shows cell survival after treatment with suboptimal (0.2 nM) levels of NGF and the left bar shows cell survival after no treatment of NGF.

FIG. 6 is a graph of cell survival in HeLa-TrkA cells for each compound (50 μM) in Series 3. The right bar shows cell survival after treatment with suboptimal (0.2 nM) levels of NGF and the left bar shows cell survival after no treatment of NGF.

FIG. 7 is a graph of cell survival in HeLa-TrkA cells for each compound (50 μM) in Series 4. The right bar shows cell survival after treatment with suboptimal (0.2 nM) levels of NGF and the left bar shows cell survival after no treatment of NGF.

FIG. 8 is a graph of cell survival in HeLa-TrkA cells for each compound (50 μM) in Series 5. The right bar shows cell survival after treatment with suboptimal (0.2 nM) levels of NGF and the left bar shows cell survival after no treatment of NGF.

FIG. 9 is a graph of cell survival in HEK293-TrkB cells for each compound (0.4 μM) in Series 1. The right bar shows cell survival after treatment with suboptimal (0.6 nM) levels of BDNF and the left bar shows cell survival after no treatment of BDNF.

FIG. 10 is a graph of cell survival in HEK293-TrkB cells for each compound (0.4 μM) in Series 2. The right bar shows cell survival after treatment with suboptimal (0.6 nM) levels of BDNF and the left bar shows cell survival after no treatment of BDNF.

FIG. 11 is a graph of cell survival in HEK293-TrkB cells for each compound (0.4 μM) in Series 3. The right bar shows cell survival after treatment with suboptimal (0.6 nM) levels of BDNF and the left bar shows cell survival after no treatment of BDNF.

FIG. 12 is a graph of cell survival in HEK293-TrkB cells for each compound (0.4 μM) in Series 4. The right bar shows cell survival after treatment with suboptimal (0.6 nM) levels of BDNF and the left bar shows cell survival after no treatment of BDNF.

FIG. 13 is a graph of cell survival in HEK293-TrkB cells for each compound (0.4 μM) in Series 5. The right bar shows cell survival after treatment with suboptimal (0.6 nM) levels of BDNF and the left bar shows cell survival after no treatment of BDNF.

FIG. 14 is a graph of cell survival in NIH3T3-TrkC cells for each compound (0.4 μM) in Series 1. The right bar shows cell survival after treatment with suboptimal (0.2 nM) levels of NT3 and the left bar shows cell survival after no treatment of NT3.

FIG. 15 is a graph of cell survival in NIH3T3-TrkC cells for each compound (0.4 μM) in Series 2. The right bar shows cell survival after treatment with suboptimal (0.2 nM) levels of NT3 and the left bar shows cell survival after no treatment of NT3.

FIG. 16 is a graph of cell survival in NIH3T3-TrkC cells for each compound (0.4 μM) in Series 3. The right bar shows cell survival after treatment with suboptimal (0.2 nM) levels of NT3 and the left bar shows cell survival after no treatment of NT3.

FIG. 17 is a graph of cell survival in NIH3T3-TrkC cells for each compound (0.4 μM) in Series 4. The right bar shows cell survival after treatment with suboptimal (0.2 nM) levels of NT3 and the left bar shows cell survival after no treatment of NT3.

FIG. 18 is a graph of cell survival in NIH3T3-TrkC cells for each compound (0.4 μM) in Series 5. The right bar shows cell survival after treatment with suboptimal (0.2 nM) levels of NT3 and the left bar shows cell survival after no treatment of NT3.

FIGS. 19A-19D are graphs of HeLa-TrkA Cell Survival Dose Response. FIG. 19A is the cell survival dose response to compound 1a(ii)ext without suboptimal neurotrophin. FIG. 19B is the cell survival dose response to compound 3a(ii) without suboptimal neurotrophin. FIG. 19C is the cell survival dose response to compound 5c(ii) with and without suboptimal neurotrophin (0.2 nM NGF). FIG. 19D is the cell survival dose response to compound 5a(iii)m with and without suboptimal neurotrophin (0.2 nM NGF).

FIG. 20A-20C are graphs of HEK293-TrkB Cell Survival Dose Response. FIG. 20A is the cell survival dose response to compound 5b(i) with and without suboptimal neurotrophin (0.6 nM BDNF). FIG. 20B is the cell survival dose response to compound 5c(i) with and without suboptimal neurotrophin (0.6 nM BDNF). FIG. 20C is the cell survival dose response to compound 5b(ii) with and without suboptimal neurotrophin (0.6 nM BDNF).

FIGS. 21A-21D are graphs of NIH3T3-TrkC Cell Survival Dose Response. FIG. 21A is the cell survival dose response to compound 3c(i) with suboptimal neurotrophin (0.2 nM NT-3).

FIG. 21B is the cell survival dose response to compound 4c(iii) with suboptimal neurotrophin (0.2 nM NT-3). FIG. 21C is the cell survival dose response to compound 5c(i) with and without suboptimal neurotrophin (0.2 nM NT-3). FIG. 21D is the cell survival dose response to compound 5c(iii) with and without suboptimal neurotrophin (0.2 nM NT-3).

FIGS. 22A-22B are graphs of the fluorescence in transfected HeLa-TrkA positive cells and non-transfected HeLa Trk negative cells (HeLa). FIG. 21A is a graph of cells treated with 5c(ii) with and without 0.2 nM of NGF (TrkA-HeLa and HeLa). FIG. 21B is a graph of cells treated with 5a(iii)m with and without 0.2 nM of NGF (TrkA-HeLa and HeLa).

FIG. 23A-23C are graphs of the fluorescence in transfected HEK293-TrkB positive cells and non-transfected HEK293 Trk negative cells (HEK293). FIG. 23A is a graph of cells treated with 5b(i) with and without 0.6 nM of BDNF (TrkB-HEK293 and HEK293). FIG. 23B is a graph of cells treated with 5c(i) with and without 0.6 nM of BDNF (TrkB-HEK293 and HEK293). FIG. 23C is a graph of cells treated with 5b(ii) with and without 0.6 nM of BDNF (TrkB-HEK293 and HEK293).

FIGS. 24A-24B are graphs of the fluorescence in transfected NIH/3T3-TrkC positive cells and non-transfected NIH/3T3 Trk negative cells (NIH/3T3). FIG. 24A is a graph of cells treated with 5c(i) with and without 0.2 nM of NT-3 (TrkC-NIH/3T3 and NIH/3T3). FIG. 24B is a graph of cells treated with 5c(iii) with and without 0.2 nM of NT-3 (TrkC-NIH/3T3 and NIH/3T3).

FIG. 25 is a graph of the fluorescence in transfected HeLa-TrkA cells where Kᵢ was determined for compound 5a(iii)m.

FIG. 26 is a graph of the fluorescence in transfected HEK293-TrkB cells where Kᵢ was determined for compounds 5b(i) and 5b(ii).

FIG. 27 is a graph of the fluorescence in transfected NIH/3T3-TrkC cells where Kᵢ was determined for compounds 5c(i) and 5c(iii).

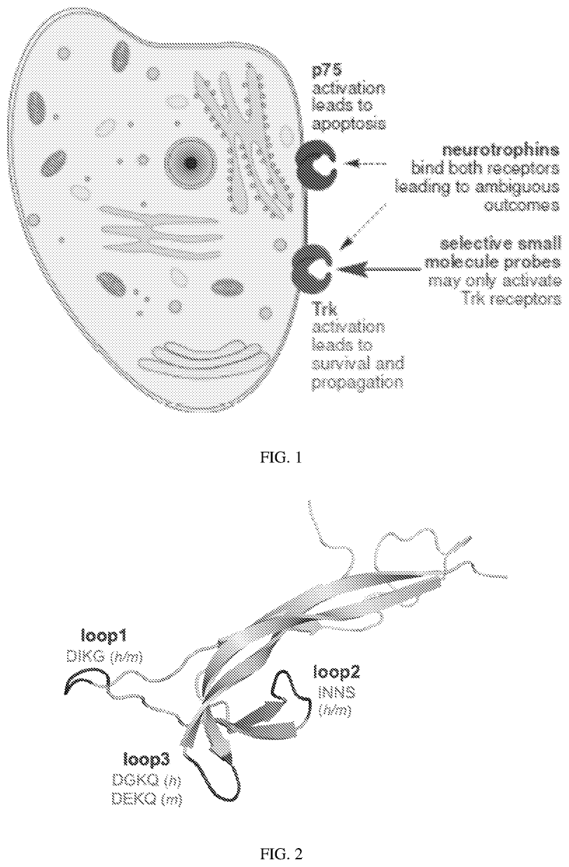

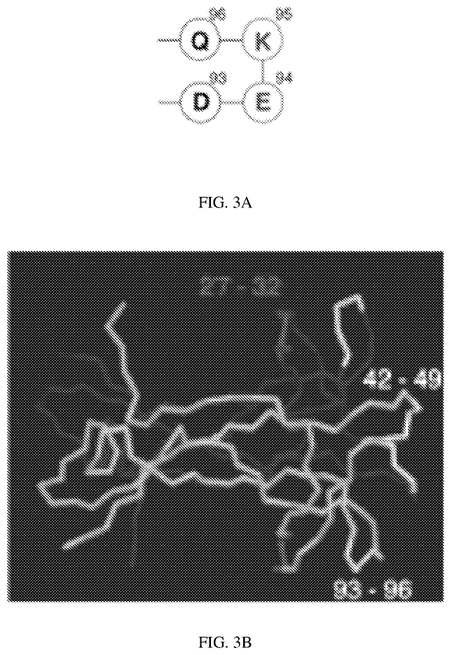

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**”, and “ [symbol] “, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by “A-*”, “A-**”, or

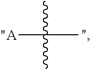

where each of”-*”, “-**”, and

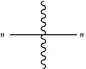

represents a bond to A and the point of covalent bond attachment to B. Alternatively, in some embodiments, the portion of A-B defined by the group or chemical structure B can be represented by “*-B”, “**-B”, or

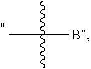

where each of “-*”, “-**”, and

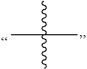

represents a bond to B and the point of covalent bond attachment to A.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

The term “cycloalkyl” refers to a saturated or partially saturated, monocyclic or polycyclic mono-valent carbocycle. The term “cycloalkylene” refers to a saturated or partially saturated, monocyclic or polycyclic divalent carbocycle. In some embodiments, it can be advantageous to limit the number of atoms in a “cycloalkyl” or “cycloalkylene” to a specific range of atoms, such as having 3 to 12 ring atoms. Polycyclic carbocycles include fused, bridged, and spiro polycyclic systems. Illustrative examples of cycloalkyl groups include monovalent radicals of the following entities, while cycloalkylene groups include divalent radicals of the following entities, in the form of properly bonded moieties:

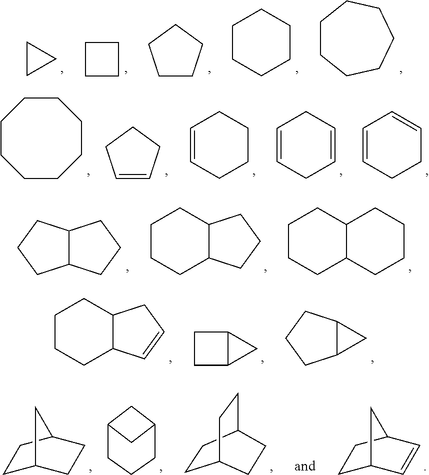

In particular, a cyclopropyl moiety can be depicted by the structural formula

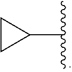 [SMILES: *C1CC1]

In particular, a cyclopropylene moiety can be depicted by the structural formula

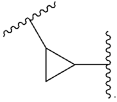 [SMILES: *C1CC1*]

It will be appreciated that a cycloalkyl or cycloalkylene group can be unsubstituted or substituted as described herein. A cycloalkyl or cycloalkylene group can be substituted with any of the substituents in the various embodiments described herein, including one or more of such substituents.

The term “heterocycloalkyl” refers to a mono-valent monocyclic or polycyclic ring structure that is saturated or partially saturated having one or more non-carbon ring atoms. The term “heterocycloalkylene” refers to a divalent monocyclic or polycyclic ring structure that is saturated or partially saturated having one or more non-carbon ring atoms. In some embodiments, it can be advantageous to limit the number of atoms in a “heterocycloalkyl” or “heterocycloalkylene” to a specific range of ring atoms, such as from 3 to 12 ring atoms (3- to 12-membered), or 3 to 7 ring atoms (3- to 7-membered), or 3 to 6 ring atoms (3- to 6-membered), or 4 to 6 ring atoms (4- to 6-membered), 5 to 7 ring atoms (5- to 7-membered), or 4 to 10 ring atoms (4- to 10-membered). In some embodiments, it can be advantageous to limit the number and type of ring heteroatoms in “heterocycloalkyl” or “heterocycloalkylene” to a specific range or type of heteroatoms, such as 1 to 5 ring heteroatoms selected from nitrogen, oxygen, and sulfur. Polycyclic ring systems include fused, bridged, and spiro systems. The ring structure may optionally contain an oxo group or an imino group on a carbon ring member or up to two oxo groups on sulfur ring members. Illustrative examples of heterocycloalkyl groups include monovalent radicals of the following entities, while heterocycloalkylene groups include divalent radicals of the following entities, in the form of properly bonded moieties:

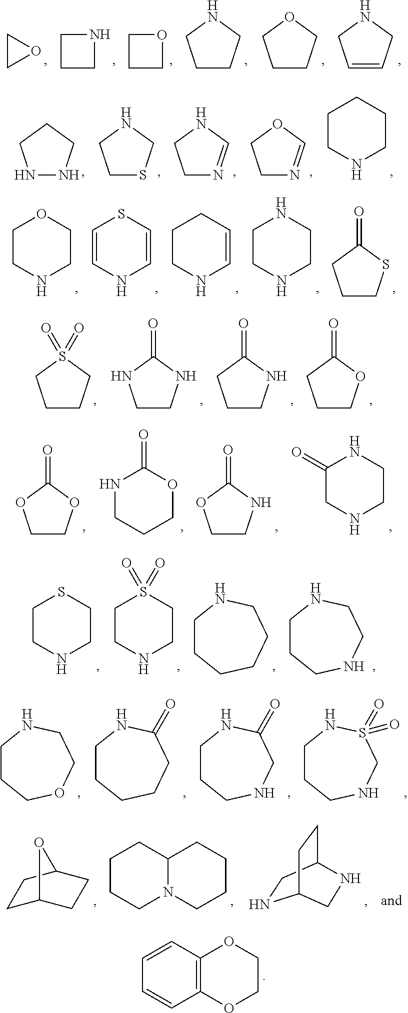

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

It will be appreciated that certain of the compounds described herein include one or more position that can exists as stereoisomers. For example, certain of the compounds described herein include one or more carbon atoms that can exist in one or more stereoisomeric arrangements. It will be appreciated that a carbon atom that can exist in stereoisomeric arrangements that is depicted without showing any stereoisomeric arrangement includes as a disclosure each of eh possible stereoisomeric arrangements. For example a carbon atom having four groups that can be priorized according to the Cahn-Ingold Prelog Rules known to one of skill in the art will be understood herein as describing no particular stereochemical definition as in the structure on the left below, and also as describing both possible stereoisomers (S) and (R) as shown below

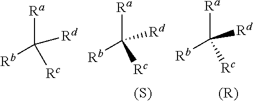

where R^{a}>R^{b}>R^{c}>R^{d} according to the Cahn-Ingold Prelog Rules.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Any disubstituent referred to herein is meant to encompass the various attachment possibilities when more than one of such possibilities are allowed. For example, reference to disubstituent -J-K-, where J K, refers herein to such disubstituent with J attached to a first substituted member and K attached to a second substituted member, and it also refers to such disubstituent with J attached to the second substituted member and K attached to the first substituted member. For example, in certain embodiments, where applicable, a compound portion -(AA)ₙ- having the sequence -DIKG-, connecting two rings, A and B, will be understood that -DIRG-, can include both of the embodiments A-DIKG-B and B-DIKG-A, provided that the groups on A and B are compatible with the terminal functional groups on the sequence -DIKG-.

The disclosure also includes pharmaceutically acceptable salts of the compounds represented by Formula (I)-(XII), preferably of those described above and of the specific compounds exemplified herein, and pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, 7-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula (I)-(XII) that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

As used herein, the term “amino acid” refers generally to alpha, beta, gamma, and longer amino acids, such as amino acids of the formula:

—N(R^{a})—(CR′R″)_{q}—C(O)—

where R^{a} is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl (alanine side chain), benzyl (phenyl alanine side chain), hydroxymethyl (serine side chain), thiomethyl (cysteine side chain), methylcarboxyl (aspartic acid side chain), ethylcarboxyl (glutamic acid side chain), guanidinopropyl (arginine side chain), and the like, and derivatives and protected derivatives thereof. The above described formula includes all stereoisomeric variations, specifically the D-configuration. The side chain for an amino acid (R′ or R″) is also described herein by other variable designations, such as R¹, R², R³, and R⁴ for convenience of being able to differentiate between the various amino side chains in a single compound. It will be understood that any of the R variable used to describe an amino acid side chain can refer to any amino acid within the present definition.

For example, one or more amino acids in the sequences described herein can be any of the 20 naturally occurring amino acids, specifically arginine (R), histidine (H), lysine (L), aspartic acid (D), glutamic acid (E), serine (S), threonine (T), asparagine (N), glutamine (Q), cysteine (C), glycine (G), proline (P), alanine (A), valine (V), isoleucine (I), leucine (L), methionine (M), phenylalanine (F), tyrosine (Y), and tryptophan (W), or the D-configuration of each. It will be appreciated that that one letter code for each amino acid in the D-configuration is the same as the one letter code for the L-configuration but in the lower case (such as D-arginine described by the one letter code (r)).

It will be further appreciated that a variety of unnatural amino acids are known in the art. Suitable unnatural amino acids include but are not limited to, selenocysteine, citrulline (Cit), hydroxyproline (Hyp), norleucine (Nle), ornithine (Orn), naphtylalanine (Nal), methionine sulfoxide, methionine sulfone, beta-alanine, α-aminobutyric acid, γ-aminobutyric acid, diaminobutyric acid, δ-aminolevulinic acid, 4-amino-benzoic acid, hydroxyproline, carboxyglutanmic acid, and the like. The above described formula includes all stereoisomeric variations, specifically the D-configuration.

### Representative Embodiments

In some embodiments, the disclosure provides a compound of the formula I

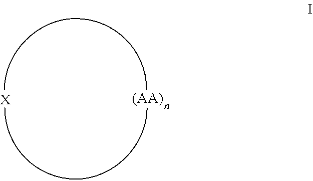

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n);
X is a divalent linking group comprising one or more of a heterocycloalkylene portion, a cycloalkylene portion, a divalent triazole portion, or a divalent dye portion; and
n is 3, 4, 5, 6, 7, or 8.

In some embodiments, the disclosure provides a compound of the formula I

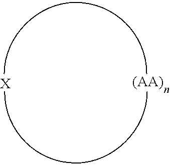

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n);
X is a divalent linking group comprising one or more of a heterocycloalkylene portion, a cycloalkylene portion, a divalent triazole portion, or a divalent dye portion; and
n is 3, 4, 5, 6, 7, or 8.

In some embodiments, the disclosure provides a compound of the formula II

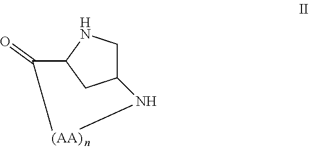

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n); and
n is 3, 4, 5, 6, 7, or 8.
In some embodiments, the disclosure provides a compound of the formula II

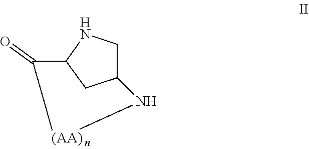

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n); and
n is 3, 4, 5, 6, 7, or 8.

In some embodiments, the disclosure provides a compound of the formula III

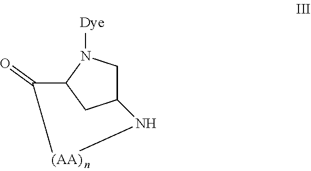

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n);
Dye is a fluorescent dye molecule; and
n is 3, 4, 5, 6, 7, or 8.

In some embodiments, the disclosure provides a compound of the formula III

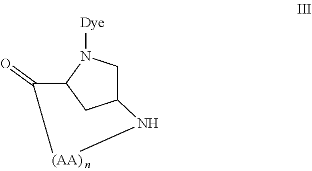

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n);
Dye is a fluorescent dye molecule; and
n is 3, 4, 5, 6, 7, or 8.

In some embodiments, the disclosure provides a compound of the formula IV

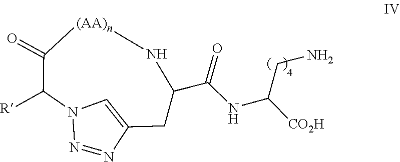

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n);
R′ is a side chain of the amino acid T, V, M, I, K, or S; and
n is 3, 4, 5, 6, 7, or 8.

In some embodiments, the disclosure provides a compound of the formula IV

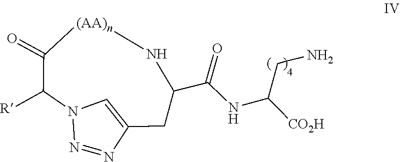

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n);
R′ is a side chain of the amino acid T, V, M, I, K, or S; and
n is 3, 4, 5, 6, 7, or 8.

In some embodiments, the disclosure provides a compound of the formula V

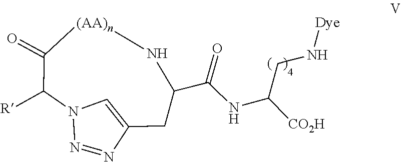

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n);
R′ is a side chain of the amino acid T, V, M, I, K, or S;
Dye is a fluorescent dye molecule; and
n is 3, 4, 5, 6, 7, or 8.

In some embodiments, the disclosure provides a compound of the formula V

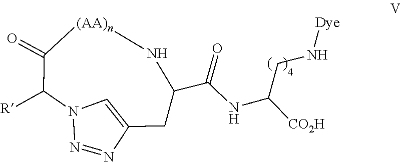

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n);
R′ is a side chain of the amino acid T, V, M, I, K, or S;
Dye is a fluorescent dye molecule; and
n is 3, 4, 5, 6, 7, or 8.

In some embodiments, disclosure provides a compound of the formula VI

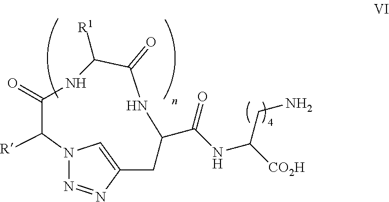

wherein R′ is an amino acid side chain as described herein, each R¹ is an amino acid side chain as described herein, and n is as described herein.

In some embodiments, disclosure provides a compound of the formula VII

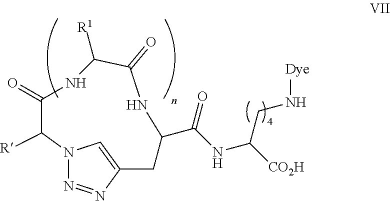

wherein R′ is an amino acid side chain as described herein, each R¹ is an amino acid side chain as described herein, and n is as described herein.

In some embodiments, the disclosure provides a compound of the formula VIII

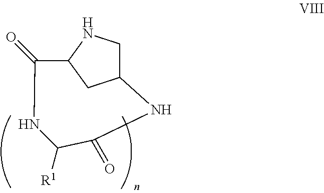

wherein each R¹ is an amino acid side chain as described herein, and n is as described herein.

In some embodiments, the disclosure provides a compound of the formula IX

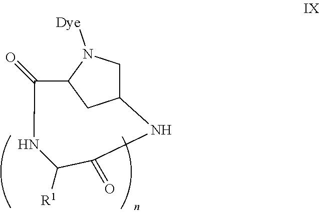

wherein each R¹ is an amino acid side chain as described herein, and n is as described herein.

In some embodiments, the disclosure provides a compound of the formula X

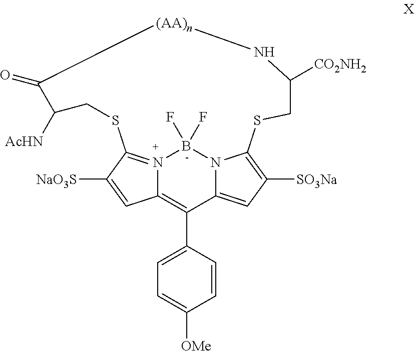

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n); and
n is 4, 5, 6, or 7.

In some embodiments, the disclosure provides a compound of the formula X

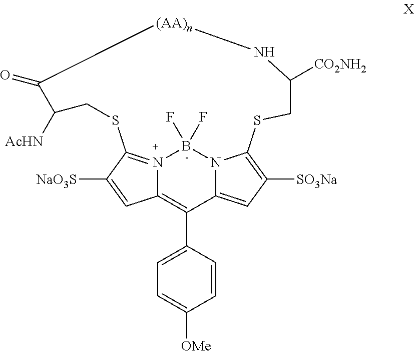

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n);
X is a divalent linking group comprising one or more of a heterocycloalkylene portion, a cycloalkylene portion, a divalent triazole portion, or a divalent dye portion; and
n is 4, 5, 6, or 7.

In some embodiments, the disclosure provides a compound of the formula XI

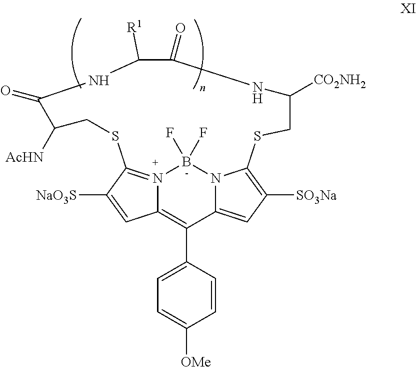

wherein each R¹ is an amino acid side chain as described herein, and n is 4, 5, 6, or 7.
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n); and
n is 4, 5, 6, or 7.

In some embodiments, the disclosure provides a compound of the formula XI

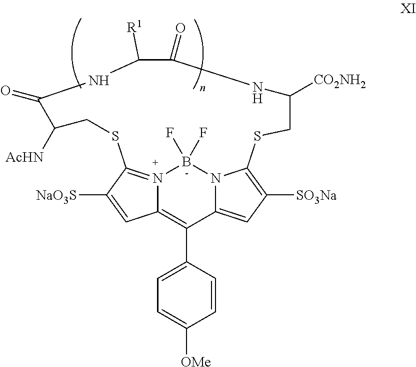

wherein each R¹ is an amino acid side chain as described herein, and n is 4, 5, 6, or 7.
(AA)ₙ is an amino acid sequence as described herein, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n); and
n is 4, 5, 6, or 7.

In one aspect, the disclosure provides a compound of the formula XII

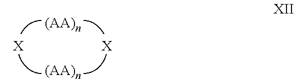

wherein
each (AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n), and the sequences of (AA)ₙ are the same;
each X is a divalent linking group comprising one or more of a heterocycloalkylene portion, a cycloalkylene portion, or a divalent triazole portion; and
n is 3, 4, 5, 6, 7, or 8.

In some embodiments, (AA)ₙ in the structures described herein defines an amino acid sequence of naturally occurring amino acids in the L- or D-configuration, and/or unnatural amino acids as described herein.

In some embodiments, one or more amino acids in the amino acid sequence is a naturally occurring amino acid selected from the group consisting of L-arginine (R), D-arginine (r), L-aspartic acid (D), D-aspartic acid (d), L-glutamic acid (E), D-glutamic acid (e), L-lysine (K), L-glycine (G), D-glycine (g), D-lysine (k), L-glutamine (Q), D-glutamine (q), L-methionine (M), D-methionine (m), L-threonine (T), D-threonine (t), L-serine (S), D-serine (s), L-cysteine (C), D-cysteine (c), L-asparagine (N), and D-asparagine (n). In some embodiments, one or more amino acids in the amino acid sequence is a naturally occurring amino acid selected from the group consisting of L-histidine (H), L-threonine (T), L-glycine (G), L-proline (P), L-alanine (A), L-valine (V), L-isoleucine (I), L-leucine (L), L-methionine (M), L-phenylalanine (F), L-tyrosine (Y), and L-tryptophan (W). In some embodiments, one or more amino acids in the amino acid sequence is a naturally occurring amino acid in the D-configuration selected from the group consisting of D-histidine (h), D-threonine (t), D-glycine (g), D-proline (p), D-alanine (a), D-valine (v), D-isoleucine (i), D-leucine (1), D-methionine (m), D-phenylalanine (f), D-tyrosine (y), and D-tryptophan (w). In some embodiments, one or more amino acids in the amino acid sequence is an unnatural amino acid selected from the group consisting of selenocysteine, citrulline (Cit), hydroxyproline (Hyp), norleucine (Nle), ornithine (Orn), naphtylalanine (Nal), methionine sulfoxide, methionine sulfone, beta-alanine, α-aminobutyric acid, γaminobutyric acid, diaminobutyric acid, δ-aminolevulinic acid, 4-amino-benzoic acid, hydroxyproline, and carboxyglutamic acid.

In some embodiments, the amino acid sequence comprises a mimetic of loop-1, loop-2, or loop-3 of a neurotrophin. In some embodiments, the neurotrophin is nerve growth factor, brain-derived neurotrophic factor, neurotrophin-3, or neurotrophin-4. It will be appreciated that the sequences of the loop regions as described herein are known in the art and are readily available to the skilled person.

In some embodiments, at least two amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine. In some embodiments, at least three amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine. In some embodiments, at least four amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine. In some embodiments, at least five amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine. In some embodiments, at least six amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine.

In some embodiments, n is 3, 4, 5, 6, 7, or 8. In some embodiments, n is 3, 4, 5, 6, or 7. In some embodiments, n is 4, 5, 6, 7, or 8. In some embodiments, n is 4, 5, 6, or 7. In some embodiments, n is 4, 5, or 6. In some embodiments, n is 3. In some embodiments, n is 4. In some embodiments, n is 5. In some embodiments, n is 6. In some embodiments, n is 7. In some embodiments, n is 8.

In some embodiments, the amino acid sequence comprises a sequence selected from the group consisting of -INS-, -snv-, -Vsn-, -DSK-, -SKk-, -sKk-, -Kks-, -ENK-, -nKV-, -vKN-, -Nne-, -ENn-, -DIKG-, -INNS-, -DGKQ-, -DEKQ-, -DMSG-, -VSKG-, -DSKK-, -DIRG-, -TQNS-, -TGNS-, -ENNK-, -DIKGK-, -NINNSVF-, -DGKQA-, -DEKQA-, -DMSGG-, -SKGQ-, -DSKKR-, -DIRGH-, -TQNSP-, -KTQNSPV-, -TQNSG-, -TGNSP-, and -ENNKLV-.

In some embodiments of the formula (I)-(IX) or (XII), the amino acid sequence comprises a sequence selected from the group consisting of -INS-, -snv-, -Vsn-, -DSK-, -SKk-, -sKk-, -Kks-, -ENK-, -nKV-, -vKN-, -Nne-, -ENn-, -DIKG-, -INNS-, -DGKQ-, -DEKQ-, -DMSG-, -VSKG-, -DSKK-, -DIRG-, -TQNS-, -TGNS-, -ENNK-, -DIKGK-, -NINNSVF-, -DGKQA-, -DEKQA-, -DMSGG-, -SKGQ-, -DSKKR-, -DIRGH-, -TQNSP-, -KTQNSPV-, -TQNSG-, -TGNSP-, and -ENNKLV-.

In some embodiments, X is a divalent linking group comprising one or more of a heterocycloalkylene portion, a cycloalkylene portion, or a divalent triazole portion. It will be appreciated that the group X in the compounds as defined herein can include additional structural fragments in addition to the fragments a heterocycloalkylene portion, a cycloalkylene portion, or a divalent triazole portion. The additional structural fragments are not particularly limited and can include amino acids, portions of amino acids, organic groups, such as an alkylene, alkenylene, alkynylene, functional groups, such as ester, amide, carbonyl, ether linkages, thiol linkages, and the like. Representative examples of additional structural pieces that can be included in the divalent linking group (X) are described herein.

In some embodiments of the formula (I), the amino acid sequence comprises a sequence selected from the group consisting of -CDEKQC-, -CINNSC-, -CDIKGC-, -CDGKQC-, -CENNKC-, -CDIRGC-, -CTGNSC-, -CTQNSC-, -CDMSGC-, -CVSKGC-, -CDSKKC-, -CDLRGC-, -CAGGSC-, -CDAQGC-, and -CDIKGC-, and the cysteine residues in the sequence are covalently attached to X via the cysteine sulfur atom. In some embodiments of the formula (I), the -OH in the carboxylic acid groups of the cysteine residues in sequence are replaced by amide or a protected amide groups. In some embodiments of the formula (I), X comprises a divalent dye portion. In some embodiments of the formula (I), the divalent dye portion is a fluorescent dye. In some embodiments of the formula (I), the divalent dye portion is a bodipy dye. In some embodiments of the formula (I), the divalent dye portion is of the formula

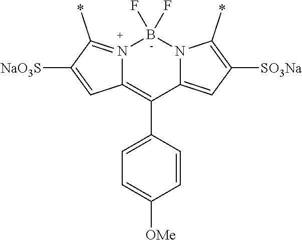

wherein each * represents a point of covalent attachment to the sulfur atom in a cysteine residue in the sequence.

In some embodiments of the formula (X) or (XI), the amino acid sequence comprises a sequence selected from the group consisting of -DEKQ-, -INNS-, -DIKG-, -DGKQ-, -ENNK-, -DIRG-, -TGNS-, -TQNS-, -DMSG-, -VSKG-, -DSKK-, -DLRG-, -AGGS-, -DAQG-, and -DIKG-. In some embodiments of the formula (X) or (XI), the -OH in the carboxylic acid groups of the cysteine residues in sequence are replaced by amide or a protected amide groups. In some embodiments of the formula (X) or (XI), X comprises a divalent dye portion. In some embodiments of the formula (X) or (XI), the divalent dye portion is a fluorescent dye. In some embodiments of the formula (X) or (XI), the divalent dye portion is a bodipy dye. In some embodiments of the formula (X) or (XI), the divalent dye portion is of the formula

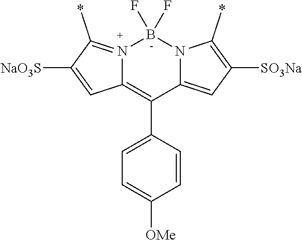

wherein each * represents a point of covalent attachment to the sulfur atom in a cysteine residue in the sequence.

In some embodiments, X comprises a heterocycloalkylene portion. In some embodiments, X comprises a pyrrolidinylene portion. In some embodiments, X is of the formula

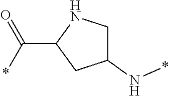 [SMILES: *NC1CNC(C(*)=O)C1]

wherein each * represents a point of covalent attachment to the rest of the compound. In some embodiments, X further comprises a dye molecule covalently attached thereto. In some embodiments, X has the formula

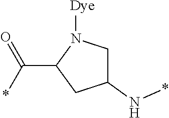

wherein each * represents a point of covalent attachment to the rest of the compound, and dye is a fluorescent dye molecule. In some embodiments, the fluorescent dye molecule is a fluorescein dye or dansyl dye.

In some embodiments, X comprises a divalent triazole portion. In some embodiments, X comprises a divalent triazole of the formula

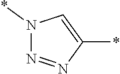 [SMILES: *c1cn(*)nn1]

wherein each * represents a point of covalent attachment to the rest of the compound. In some embodiments, X comprises the structure

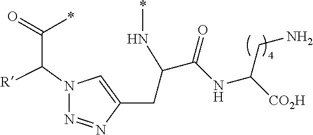

wherein each * represents a point of covalent attachment to the rest of the compound. In some embodiments, X further comprises a dye molecule covalently attached thereto.

In some embodiments, X has the formula

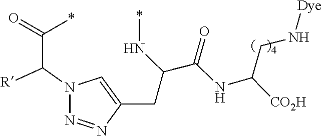

wherein each * represents a point of covalent attachment to the rest of the compound, and dye is a fluorescent dye molecule. In some embodiments, the fluorescent dye molecule is a fluorescein dye or dansyl dye.

In some embodiments, the disclosure provides to a linear compound of the formula XIII

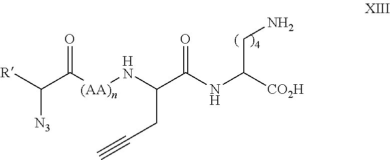

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), D-asparagine (n), L-leucine (L), D-leucine (1), L-isoleucine (I), D-isoleucine (i), L-threonine (T), D-threonine (t), L-glycine (G), D-glycine (g), L-valine (V), D-valine (v), L-phenylalanine (F), D-phenylalanine (f), L-proline (P), and D-proline (p);
R′ is a side chain of the amino acid T, V, M, I, K, or S; and
n is 3, 4, 5, 6, 7, or 8.

In some embodiments, the disclosure provides to a linear compound of the formula XIV

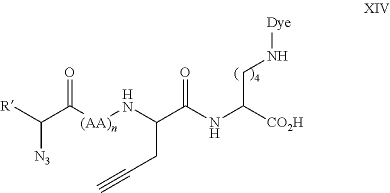

wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), D-asparagine (n), L-leucine (L), D-leucine (1), L-isoleucine (I), D-isoleucine (i), L-threonine (T), D-threonine (t), L-glycine (G), D-glycine (g), L-valine (V), D-valine (v), L-phenylalanine (F), D-phenylalanine (f), L-proline (P), and D-proline (p);
R′ is a side chain of the amino acid T, V, M, I, K, or S;
Dye is a fluorescent dye molecule; and
n is 3, 4, 5, 6, 7, or 8.

In some embodiments, disclosure provides to a linear compound of the formula XV

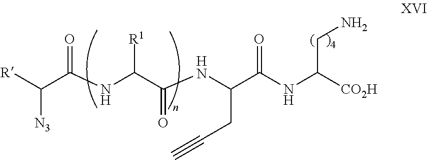

wherein R′ is an amino acid side chain as described herein, each R¹ is an amino acid side chain as described herein, and n is as described herein.

In some embodiments, disclosure provides to a linear compound of the formula XVI

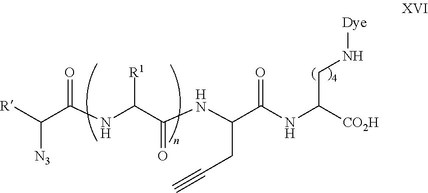

wherein R′ is an amino acid side chain as described herein, each R¹ is an amino acid side chain as described herein, and n is as described herein.

Those skilled in the art will recognize that the species listed or illustrated herein are not exhaustive, and that additional species within the scope of these defined terms may also be selected.

### Pharmaceutical Compositions and Uses

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the disclosure are sterile compositions. Pharmaceutical compositions may be prepared using compounding techniques known or that become available to those skilled in the art.

Sterile compositions are also contemplated by the disclosure, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the disclosure may be administered by a suitable route of delivery, such as oral, parenteral, rectal, nasal, topical, or ocular routes, or by inhalation. Preferably, the compositions are formulated for intravenous or oral administration.

For oral administration, the compounds the disclosure may be provided in a solid form, such as a tablet or capsule, or as a solution, emulsion, or suspension. To prepare the oral compositions, the compounds of the disclosure may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present disclosure are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the disclosure may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human patient.

It will be appreciated that the compounds described herein can be used for treating disease. In some embodiments, the disease is one in which cell survival is mediated by one or more of TrkA, TrkB, or TrkC. In some embodiments, the compounds and pharmaceutical compositions described herein can be administered to a subject in need of treatment to stimulate one of more of TrkA, TrkB, or TrkC to treat a disease. Exemplary diseases include neurological diseases or eye diseases. In some embodiments, the disease is an eye disease. In some embodiments, the disease is glaucoma, dry eye disease, retinitis pigmentosa, or neurotrophic keratitis.

In treatment methods according to the disclosure, an “effective amount” means an amount or dose sufficient to generally bring about the desired therapeutic benefit in subjects needing such treatment. Effective amounts or doses of the compounds of the disclosure may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### Chemical Synthesis Methods

The following examples are offered to illustrate but not to limit the disclosure. One of skill in the art will recognize that the following synthetic reactions and schemes may be modified by choice of suitable starting materials and reagents in order to access other compounds of Formula (I)-(XII).

### General Procedures for the Synthesis of Series 1 Compounds

### Fmoc-Lys(dansyl)-OH:

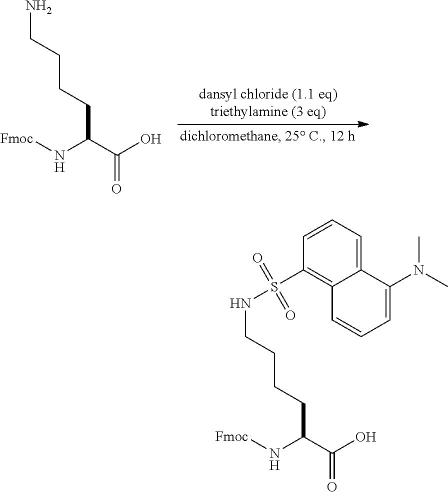

Synthesis of dienylated lysine was adapted from (Burgess, K.; Jacutin, S. E.; Lim, D.; Shitangkoon, A., J. Org. Chem. 1997, 62, 5165-5168). Fmoc-Lys-OH (5 mmol) was dissolved in 50 mL of dry dichloromethane under N₂. Triethylamine (3 eq, 15 mmol) followed by dansyl chloride (1.1 eq, 5.5 mmol) were added and the mixture stirred under nitrogen for 12 hours. The solution was neutralized with glacial acetic acid (3 eq, 15 mmol) followed by purification via flash column chromatography starting with 100% hexanes, gradually increasing to 1:1 hexanes:ethyl acetate.

### α-Azido Acids:

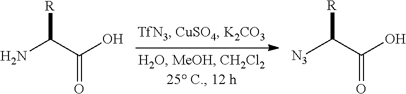

α-Azido acids were prepared as per previously reported in the literature.¹ Briefly, preparation was achieved by dissolving the amino acid, CuSO₄, and K₂CO₃ in a 1:2 H₂O:MeOH mixture. Triflic azide in dichloromethane was added, and the mixture stirred overnight. The azido acids were purified via a buffered extraction to remove the sulfonamide byproduct.

### Method 1: General Procedure for Peptide Synthesis and Cyclization of Series 1 Compounds

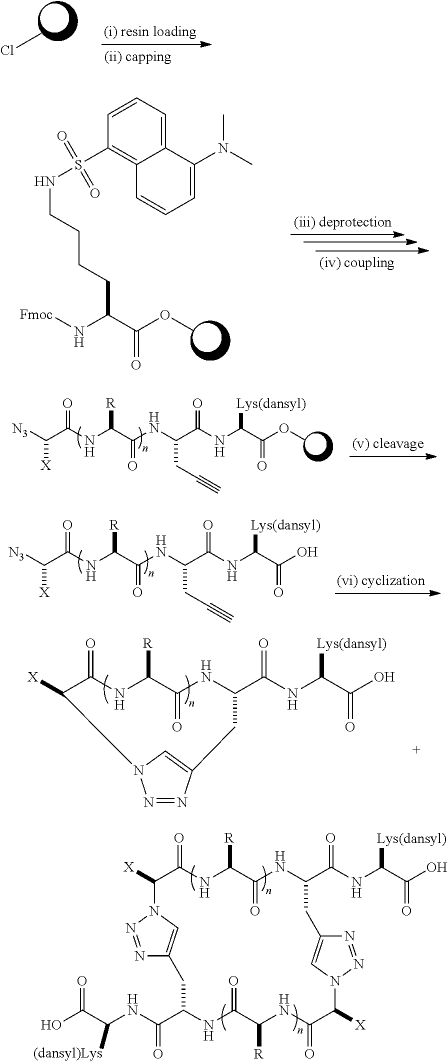

Series 1 compounds were prepared via standard Fmoc synthesis procedure on a Liberty Blue peptide synthesizer on 2-chlorotrityl resin, followed by cleavage and cyclization in solution (See “Turner, R. A.; Oliver, A. G.; Lokey, R. S., Org. Lett. 2007, 7, 5011-5014”).

(i) 2-Chlorotrityl resin was pre-swelled in dichloromethane (DCM), then 2.5 mL of 0.2 M Fmoc-Lys(dansyl)-OH in dimethylformamide (DMF) was added to the resin followed by 2.5 mL of 0.5 M diisopropylethylamine (DIEA) in DCM. The mixture was microwaved at 50° C. for 30 minutes, washed twice with DMF, then the loading cycle repeated one more time. (ii) Free Cl groups on the resin were capped using 2 cycles of 8 mL of 1:3:7 DIEA:methanol:DCM for 10 minutes at room temperature.

(iii) and (iv) The Fmoc groups were deprotected with 20% piperidine in DMF at 600° C. for 4 minutes, then washed 4 times with DMF. 2.5 mL of 0.2 M Fmoc-amino acid (or amino azide X as the final coupling) in DMF, 1 mL of 0.45 M hexafluorophosphate azabenzotriazole tetramethyl uranium (HATU) in DMF, and 0.5 mL of 0.5 M DIEA in DMF were added to the reaction vessel and coupled at 500° C. for 8 minutes. Coupling cycles were repeated until the desired linear peptide was complete.

(v) Protected peptides were cleaved from the resin using 5% trifluoroacetic acid (TFA) in DMF and the solvent removed in vacuo.

(vi) Peptides were cyclized at a concentration of 1 mM in DMF using copper (II) sulfate-pentahydrate (0.2 eq), sodium ascorbate (0.5 eq), and DIEA (5 eq). Argon gas was bubbled through the solution for 15 minutes and the reaction allowed to run overnight.

Cyclized products were deprotected for 2 hours with a solution of 95% TFA, 2.5% H₂O, 2.5% triisopropylsilane. Solvent was removed with a stream of nitrogen, then the peptide product precipitated from cold ether and purified via preparative HPLC.

Dimer products were purified and collected as side-products of the cyclization reaction and tested in addition to the cyclic monomers. Purity and identity were determined by analytical HPLC, LCMS, and HRMS.

### Series 1 Compounds Prepared by Method 1

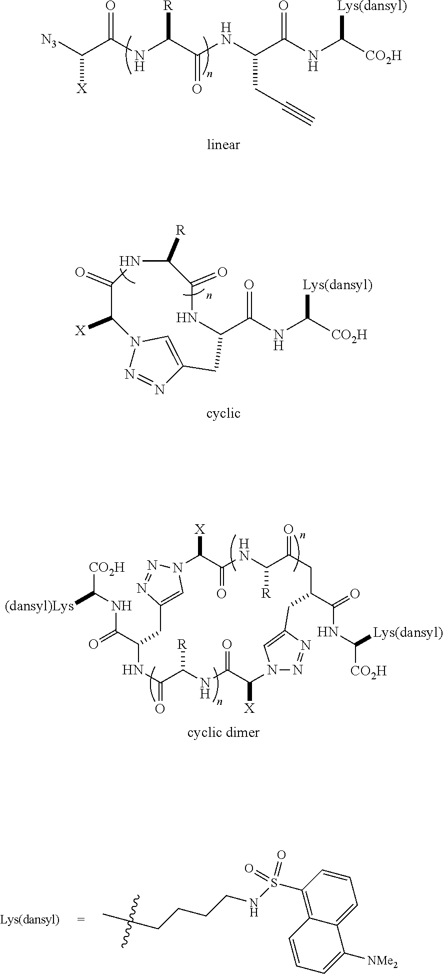

TABLE 1
Series 1 Comounds

| | | | m/z calc. | m/z found |
|---|---|---|---|---|
| Name | X^{a} | Rₙ | [M + H] | [M + H] |
| 1a(i) | T | DIKGK | 1143.56 | 1143.4 |
| 1a(ii)ext | V | NINNSVF | 694.82^{b} | 694.8^{b} |
| 1a(iii) | M | DGKQA | 1131.47 | 1131.3 |
| 1a(iii)mut | M | DEKQA | 1203.49 | 1203.3 |
| 1a(iii)mouse | T | DEKQA | 1173.50 | 1173.3 |
| 1b(i) | V | DMSGG | 1047.40 | 1047.2 |
| 1b(ii) | V | SKGQ | 1000.47 | 1000.3 |
| 1b(ii)lin | V | SKGQ | 1000.47 | 1000.9 |
| 1b(iii) | M | DSKKR | 1246.58 | 1247.4 |
| 1c(i) | I | DIRGH | 596.79^{b} | 596.8^{b} |
| 1c(i)lin | I | DIRGH | 596.79^{b} | 598.8^{b} |
| 1c(ii)dim | K | TONSP | 1157.02^{b} | 1157.3^{b} |
| 1c(ii)ext | I | KTQNSPV | 1368.67 | 1369.3 |
| 1c(ii)mutlin | K | TQNSG | 1116.49 | 1116.3 |
| 1c(ii)mutdim | K | TQNSG | 1116.99^{b} | 1116.6^{b} |
| 1c(ii)mouse | K | TGNSP | 1085.48 | 1087.3 |
| 1c(ii)mousedim | K | TGNSP | 1085.98^{b} | 1085.7^{b} |
| 1c(iii) | S | ENNKLV | 1285.60 | 1286.4 |

^{a}The one letter amino acid code denotes the side chain of the respective amino acid
^{b}Indicative of a m/z of [M + 2H]/2

### General Procedures for the Synthesis of Series 2 Compounds

All compounds of Series 2 were prepared as previously done in the literature (See “Zaccaro, M. C.; Lee H. B.; Pattarawarapan, M.; Xia, Z.; Caron, A.; L'Heureux, P. J.; Bengio, Y.; Burgess, K.; Saragovi, H. U., Chem. Biol. 2005, 12, 1015-1028”).

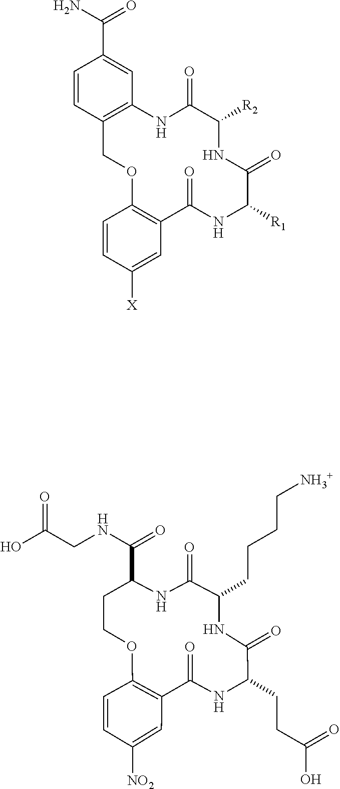

TABLE 2
Series 2 Comounds

| Name | R¹ | R² | X |
|---|---|---|---|
| 2a(i)IK | Ile | Lys | NH₂ |
| 2a(i)′KG* | Lys | Gly | NHSO₂Me |
| 2a(iii)GK | Gly | Lys | NH₂ |
| 2c(i)RG | Arg | Gly | NH₂ |
| 2c(i)′IR* | Ile | Arg | NHSO₂Me |
| 2c(i)′RG* | Arg | Gly | NHSO₂Me |
| D3 | Glu | Lys | NO₂ |

### General Procedures for the Synthesis of Series 3 and 4 Compounds

### Method 2: General Procedure for Peptide Synthesis and Cyclization of Series 3 and 4 Compounds

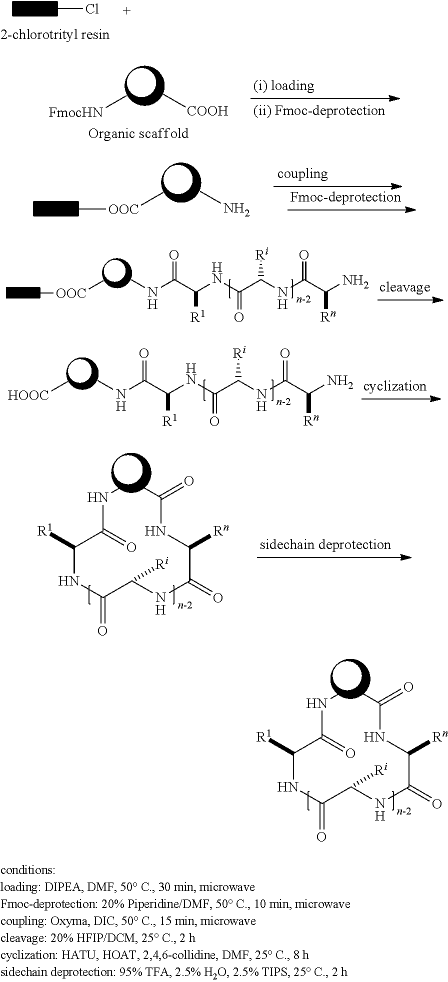

The organic scaffold (N-Boc-cis-4-N-Fmoc-amino-L-proline, 0.48 mmol) was dissolved in DMF (4 mL) and DIPEA (1.2 mmol) was added. Half of the solution was added to a syringe with 2-chlorotrityl resin (0.2 mmol) and the mixture was microwaved at 50° C. for 30 min. Used solution was drained and the left solution was added. Mixture was microwaved at 50° C. for 30 min and solution inside syringe was drained. Loaded resin was washed by DMF (2 mL) for 3 times. 20% Piperidine/DMF (2 mL) was added to the syringe and microwaved at 50° C. for 10 min. Used solution was drained. The same procedure was repeated once to fully deprotect the Fmoc group. The resin was washed by DMF (2 mL) for 3 times before the next step.

Following couplings and deprotection of regular Fmoc-protected amino acids were implemented on a peptide synthesizer (liberty blue, CEM). Reaction scale was set as 0.25 mmol. As for coupling, Oxyma (activator base, 1.0 M, 1 mL), DIC (activator, 0.5 M, 2 mL), Fmoc-amino acid (0.2 M, 5 mL) and resin was mixed and microwaved at 50° C. for 15 min. Used solution was drained and resin was washed by DMF (2 mL) for 3 min. As for deprotection, 20% Piperidine/DMF (5 mL) was added into the reaction vessel and microwaved at 50° C. for 10 min. Used solution was drained and resin was washed by DMF (2 mL) for 3 min.

After repeated cycles of coupling and deprotection, resin-linked linear peptide was transferred back to a syringe. 20% HFIP/DCM (3 mL) was added and shaken for 3 h to cleave the peptide from resin. Solution was stored in a round bottle flask (250 mL) and solvents (HFIP, DCM) was removed by constant air flow. HATU (0.6 mmol), HOAT (0.6 mmol), 2,4,6-collidine (0.6 mmol) and DMF (60 mL) were added to the flask (See “Pelletier, J. C. and Lundquist, J. T., Org. Lett. 2001, 3, 781-783”). The mixture was stirred for 8 h to cyclize the linear peptide. After the reaction was completed, DMF was removed by high-vacuum rotavapor. Water/acetonitrile (2-3 mL) were added to dissolve the remaining oils and the sidechain-protected cyclic peptide is purified by prepHPLC. The purified cyclic peptide is dissolved in 95% TFA/2.5% H₂O/2.5% TIPS and stirred for 3 h to deprotect all remaining protecting groups. Diethyl ether was added and deprotected compound was precipitated after centrifuging for 5 min. Liquid was poured and the precipitate was dissolved by water/acetonitrile (2-3 mL). Crude peptide was further purified by prepHPLC to yield pure product.

Retention times (rt) were from analytical HPLC runs using a Zorbax SB-C18 column (Agilent) with a 20 minute gradient between 5% solvent A (99.9% water, 0.1% TFA) and 95% solvent B (99.9% acetonitrile, 0.1% TFA), and 95% solvent A and 5% solvent B. Expected masses of peptides were calculated in ChemDraw. MS were obtained from ESI-MS.

### Series 3 Compounds Prepared by Method 2

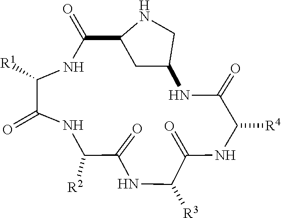

TABLE 3
Series 3 Comounds

| Name | R¹ | R² | R³ | R⁴ | HPLC (rt) | m/z found |
|---|---|---|---|---|---|---|
| 3a(i) | Asp | Ile | Lys | Gly | 9.044 | 263.6522 |
| 3a(ii) or A1 | Ile | Asn | Asn | Ser | 7.738 | 541.2718 |
| 3a(iii) | Asp | Gly | Lys | Gln | 5.496 | 542.2560 |
| 3a(iii)mouse | Asp | Glu | Lys | Gln | 5.806 | 613.2924 |
| 3b(i) | Asp | Met | Ser | Gly | 8.828 | 503.1906 |
| 3b(ii) | Val | Ser | Lys | Gly | 8.397 | 484.2869 |
| 3b(iii) | Asp | Ser | Lys | Lys | 5.830 | 571.3184 |
| 3c(i) or Pan | Asp | Ile | Arg | Gly | 9.449 | 277.6553 |
| 3c(ii) | Thr | Gln | Asn | Ser | 5.170 | 543.2515 |
| 3c(ii)mouse | Thr | Gly | Asn | Ser | 5.212 | 472.2146 |
| 3c(iii) | Glu | Asn | Asn | Ser | 5.555 | 299.6502 |

### Series 4 Compounds Prepared by Method 2

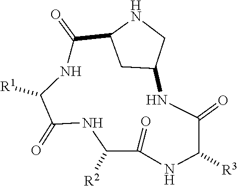

TABLE 4
Series 4 Comounds

| Name | R¹ | R² | R³ | HPLC (rt) | m/z found |
|---|---|---|---|---|---|
| 4a(ii)INS | Ile | Asn | Ser | 5.964 | 427.2301 |
| 4a(ii)snv | D-Ser | D-Asn | D-Val | 7.780 | 413.2143 |
| 4a(ii)Vsn | Val | D-Ser | D-Asn | 7.097 | 413.2143 |
| 4b(iii)DSK | Asp | Ser | Lys | 5.384 | 443.2249 |
| 4b(iii)SKk | Ser | Lys | D-Lys | 4.647 | 228.6501 |
| 4b(iii)sKk | D-Ser | Lys | D-Lys | 5.568 | 228.6502 |
| 4b(iii)Kks | Lys | D-Lys | D-Ser | 5.586 | 228.6502 |
| 4c(iii)ENK | Glu | Asn | Lys | 5.443 | 242.6294 |
| 4c(iii)nKV | D-Asn | Lys | Val | 9.025 | 454.2770 |
| 4c(iii)vkN | D-Val | Lys | Asn | 7.508 | 454.2773 |
| 4c(iii)Nne | Asn | D-Asn | D-Glu | 5.312 | 470.1998 |
| 4c(iii)ENn | Glu | Asn | D-Asn | 4.944 | 470.1993 |

### General Procedures for the Synthesis of Series 5 Compounds

### BODIPY Dye Synthesis

The BODIPY dye was synthesized following the general procedure found in the literature (See “Li, L.; Han, J.; Nguyen, B.; Burgess, K., J. Org. Chem. 2008, 73, 1963-1970”).

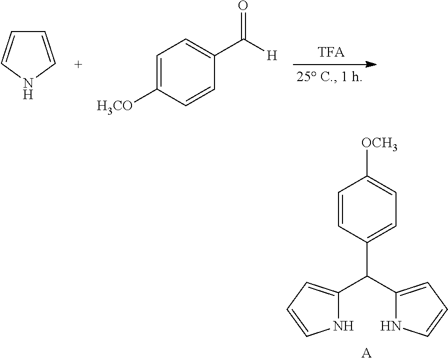

Pyrrole (142 mL, 2.6 mol, 25 eq) and 4-methoxybenzaldehyde (10 mL, 82.2 mmol, 1 eq) were added to a 500 mL round-bottomed flask and degassed with a stream of Ar gas for 5 min. Trifluoroacetic acid (0.63 mL, 8.2 mmol, 0.1 eq) was added to the reaction mixture. The reaction was stirred under argon at 25° C. for 1 h. The excess pyrrole was removed under reduced pressure. The residue was purified via silica column chromatography using dichloromethane to give a yellow solid (7.74 g, 38%). ¹H NMR (400 MHz, CDCl₃) δ (ppm)=7.90 (br, 2H), 7.17 (d, J=8.56 Hz, 2H), 6.90 (d, J=8.68 Hz, 2H), 6.71-6.69 (m, 2H), 6.21-6.19 (m, 2H), 5.96-5.95 (m, 2H), 5.44 (s, 1H), 3.83 (s, 3H) ppm. ¹³C NMR (100 MHz, CDCl₃) δ (ppm)=158.9, 134.3, 132.9, 129.4, 117.1, 114.0, 108.4, 107.0, 55.3, 43.2 ppm. HRMS(ESI+) calcd. for C₁₆H₁₅N₂O⁺ [M−H]⁺ 251.1179, found 251.1175.

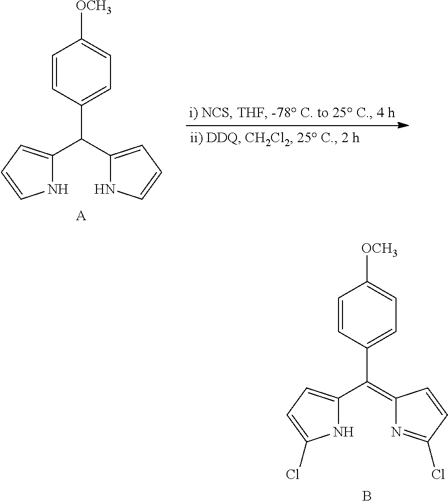

A solution of A (7.74 g, 30.6 mmol, 1 eq) in 220 mL of tetrahydrofuran was purged with Ar gas and cooled to −78° C. A suspension of N-chlorosuccinimide (8.2 g, 61.3 mmol, 2 eq) in 80 mL of tetrahydrofuran was added to the cooled solution. The reaction mixture was stirred at -78° C. for 1 h and warmed to 25° C., then stirred for an additional 3 h. Water (100 mL) was added to the mixture. After extraction with CH₂Cl₂ (3×100 mL), the combined organic layers were dried over anhydrous MgSO₄, filtered, and the solution was evaporated to dryness. The residue was immediately used without further purification.

DDQ (8.0 g, 35.2 mmol, 1.2 eq) was added to the solution of the intermediate dichloro-dipyrromethane in 350 mL of dichloromethane. The mixture was stirred at 25° C. for 2 h. After evaporation of the solvent, the residue was purified by silica column chromatography using 5% EtOAc in CH₂Cl₂ to afford a red solid (7.0 g, 63% for 2 steps). ¹H NMR (400 MHz, CDCl₃) δ (ppm)=7.29, (d, J=8.64 Hz, 2H), 6.88 (d, J=8.68 Hz, 2H), 6.49, (d, J=4.24 Hz, 2H), 6.17 (d, J=4.24 Hz, 2H), 3.80 (s, 3H) ppm. ¹³C NMR (100 MHz, CDCl₃) δ (ppm)=160.8, 141.4, 140.1, 138.5, 132.5, 130.1, 127.8, 116.7, 113.5, 55.4 ppm. HRMS(ESI+) calcd. for C₁₆H₁₃Cl₂N₂O⁺ [M−H]⁺ 319.0399, found 319.0396.

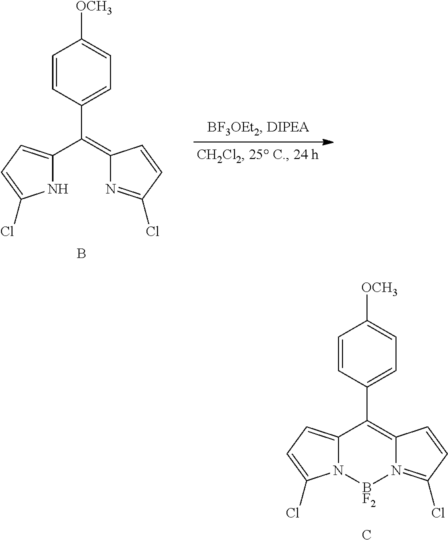

A solution of compound B (7 g, 22.0 mmol, 1 eq) and N,N-diisopropylethylamine (22.9 mL, 132 mmol, 6 eq) in 200 mL of dry dichloromethane was stirred under argon atmosphere at 25° C. for 10 min. Then boron trifluoride diethyletherate (27.6 mL, 219 mmol, 10 eq) was added slowly over 10 min. The resulting solution was stirred at 25° C. for 24 h. Then the solution was washed with water (3×100 mL), dried over anhydrous MgSO₄, filtered and evaporated to dryness. The residue was purified by silica column chromatography using 1% EtOAc in CH₂Cl₂ to afford a brown solid (3.2 g, 40%). ¹H NMR (400 MHz, CDCl₃) δ (ppm)=7.37 (d, J=8.7 Hz, 2H), 6.96 (d, J=8.7 Hz, 2H), 6.80 (d, J=4.3 Hz, 2H), 6.36 (d, J=4.3 Hz, 2H), 3.83 (s, 3H) ppm. ¹³C NMR (100 MHz, CDCl₃) δ (ppm)=162.2, 144.2, 144.1, 133.7, 132.3, 131.4, 124.8, 118.6, 114.2, 55.6 ppm. HRMS(ESI+): m/z caled for C₁₆H₁₂BCl₂F₂N₂O⁺ [M−H]⁺ 367.0382, found 367.0378.

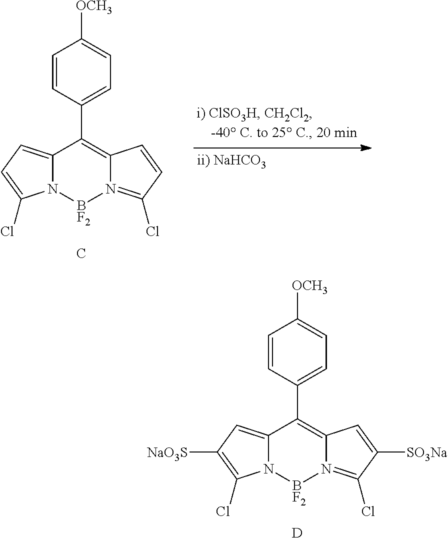

A solution of chlorosulfonic acid (0.4 mL, 5.99 mmol, 2.2 eq) in 5 mL of dichloromethane was added dropwise to a solution of compound C (1 g, 2.73 mmol, 1 eq) in 45 mL of dichloromethane over 10 min under argon at −40° C. The solution was then warmed slowly to 25° C. The product precipitated from the reaction mixture and was isolated via filtration. The solids were then dissolved in water and neutralized with NaHCO₃ (0.51 g, 6.0 mmol, 2 eq). The aqueous solution was lyophilized to afford a brown solid (800 mg, 53%). The compound was further purified via preparative HPLC. ¹H NMR (400 MHz, D₂O) δ (ppm)=7.53 (d, J=8.7 Hz, 2H), 7.31 (s, 2H), 7.13 (d, J=8.7 Hz, 2H), 3.92 (s, 3H) ppm. ¹³C NMR (100 MHz, D₂O) δ (ppm)=163.0, 148.7, 140.4, 133.5, 133.3, 131.3, 123.9, 114.7, 55.8 ppm. HRMS(ESI−) calcd for C₁₆H₉BCl₂F₂N₂O₇S₂²⁻ [M−2Na]²⁻ 261.9639, found 261.9652.

### Method 3: General Procedure for Peptide Synthesis and Cyclization of Series 5 Compounds

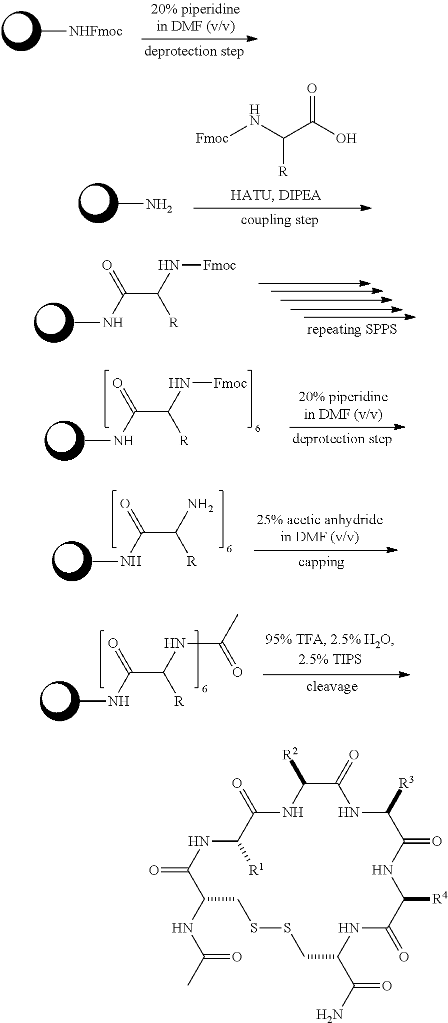

The peptides were synthesized according to SPPS using Fmoc/tBu strategy. Resin beads (TentaGel® S RAM, 0.23 mmol/g, 1 g) were swelled in DMF in 10 mL-fritted syringe for 30 min and removed the solvent by vacuum filtration then. The deprotection step, 20% piperidine in DMF (v/v) was stirred for 10 min and wash out for 3 times by vacuum filtration. The resin was washed with DMF for 5 times before amino acid coupling to resin. The coupling step, mixed solution of amino acid (4 eq, 0.92 mmol), HATU (3 eq, 0.69 mmol) and DIPEA (6 eq, 1.38 mmol) in DMF was stirred in resin for 30 min and removed by vacuum filtration and then washed with DMF 3 times. Next amino acid was repeated the cycle starting from deprotection to coupling step for each of the subsequent amino acids using the following Fmoc-amino acid derivatives: Fmoc-Ile-OH (I), Fmoc-Glu(OtBu)-OH (E), Fmoc-Met-OH (M), Fmoc-Leu-OH (L), Fmoc-Lys(Boc)-OH (K), Fmoc-Arg(Pbf)-OH (R), Fmoc-Asn(Trt)-OH (N), Fmoc-Ser(tBu)-OH (S), Fmoc-Ala-OH (A), Fmoc-Gln(Trt)-OH (Q), Fmoc-Val-OH (V), Fmoc-Thr(tBu)-OH (T), Fmoc-Asp(OtBu)-OH (D), and Fmoc-Cys(Trt)-OH (C). The deprotection step was performed to remove Fmoc before capped with solution of 25% acetic anhydride in DMF (v/v) for 15 min and then removed the solvent by vacuum filtration. DMF was washed out by dichloromethane for 5 times.

The acid cleavage cocktail (95% TFA, 2.5% H₂O, and 2.5% TIPS) was prepared and added to the dried peptide resin, stirred gently for 1 h to cleavage side chain protecting groups and cleavage the peptide from the resin. The peptide was collected from the drained acid cleavage cocktail for 2 times. The collected peptide was purged by N₂ gas to remove TFA and worked-up and precipitated using cold diethyl ether. To isolate peptide solution, the crude peptide was spined down by centrifugation (2400 rpm, 5 min) and washed with cold diethyl ether twice. The crude peptide was dissolved in 10% ACN in H₂O (v/v) and lyophilized to make crude peptide cleaner. Purification of the crude product, crude peptide was dissolved in 0.5 ml ACN and 2.0 ml of 0.1% aqueous TFA, filtrated by syringe filter 13 mm before injected to preparative HPLC with 30% ACN gradient in H₂O system for 20 min. Collect the fractions corresponding to the main peak and remove the ACN by evaporation at reduced pressure. The aqueous solution is finally lyophilized and checked by analytical HPLC.

### General Method of Dye Insertion

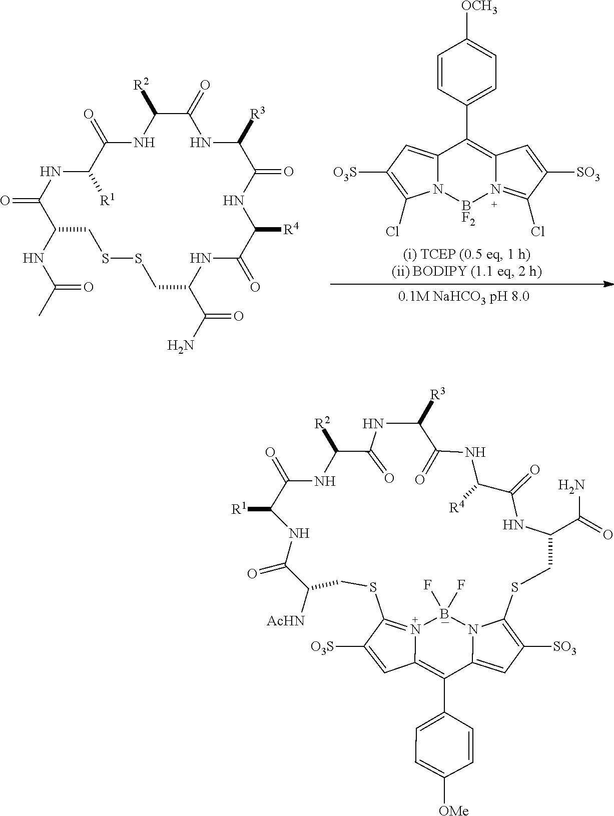

The peptide (0.02 mmol) was dissolved in 19.6 mL of 0.1 M NaHCO₃ (pH 8.0) and purged by N₂ gas for 30 min to remove any oxidizing agent. Solution of TCEP (0.5 eq, 0.01 mmol) in water (0.5 mL) was added and stirred for 1 h to reduce disulfide to thiols. Then, solution of BODIPY (1.1 eq, 0.022 mmol) in water (0.5 mL) was added to the mixture and stirred at 25° C. for 2 h. The crude product was lyophilized overnight. For purification, crude product was dissolved in H₂O, filtrated by syringe filter 13 mm before injected to preparative HPLC with 30% ACN gradient in H₂O system for 20 min. Collect the fractions corresponding to the main peak. After lyophilized, the product is finally obtained in red solid and checked the purity by analytical HPLC and characterized by ¹H NMR, TOCSY-NMR and high-resolution mass spectrometry (HRMS).

### Series 5 Compounds Prepared by Method 3

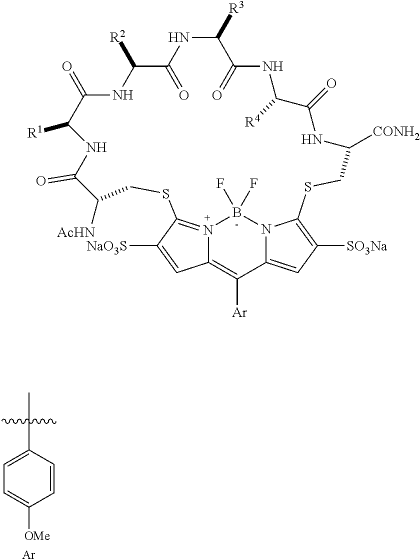

TABLE 5
Series 5 Compounds

| com- | sequence | | | | | neurotrophin | |
|---|---|---|---|---|---|---|---|
| pound | C-(AA¹⁻⁴)-C | R¹ | R² | R³ | R⁴ | organism* | loop |
| 5a(i) | -DIKG- | 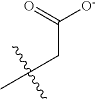 [SMILES: *CC(=O)[O-]] | 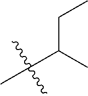 [SMILES: *C(C)CC] | 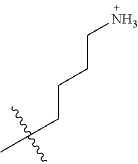 [SMILES: *CCCCN] | 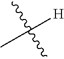 [SMILES: *[H]] | hNGF | 1 |
| 5b(i) | -DMSG- | 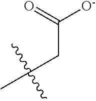 [SMILES: *CC(=O)[O-]] | 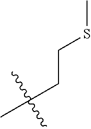 [SMILES: *CCSC] | 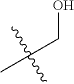 [SMILES: *CO] | 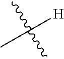 [SMILES: *[H]] | hBDNF | 1 |
| 5c(i) | -DIRG- | 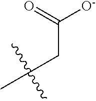 [SMILES: *CC(=O)[O-]] | 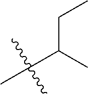 [SMILES: *C(C)CC] | 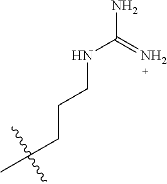 [SMILES: *CCCNC(=N)N] | 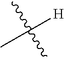 [SMILES: *[H]] | hNT-3/ mNT-3 | 1 |
| 5d(i) | -DLRG- | 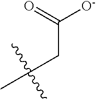 [SMILES: *CC(=O)[O-]] | 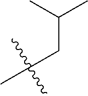 [SMILES: *CC(C)C] | 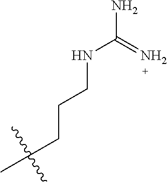 [SMILES: *CCCNC(=N)N] | 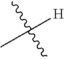 [SMILES: *[H]] | hNT-4 | 1 |
| 5a(ii) | -INNS- | 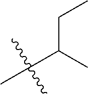 [SMILES: *C(C)CC] | 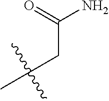 [SMILES: *CC(N)=O] | 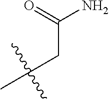 [SMILES: *CC(N)=O] | 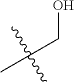 [SMILES: *CO] | hNGF | 2 |
| 5b(ii) | -VSKG- | 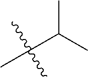 [SMILES: *C(C)C] | 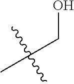 [SMILES: *CO] | 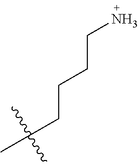 [SMILES: *CCCCN] | 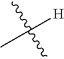 [SMILES: *[H]] | hBDNF | 2 |
| 5c(ii) | -TQNS- | 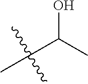 [SMILES: *C(C)O] | 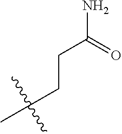 [SMILES: *CCC(N)=O] | 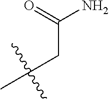 [SMILES: *CC(N)=O] | 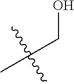 [SMILES: *CO] | hNT-3 | 2 |
| 5c(ii)m | -TGNS- | 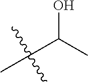 [SMILES: *C(C)O] | 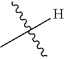 [SMILES: *[H]] | 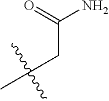 [SMILES: *CC(N)=O] | 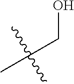 [SMILES: *CO] | mNT-3 | 2 |
| 5d(ii) | -AGGS- | 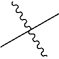 | 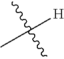 [SMILES: *[H]] | 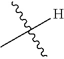 [SMILES: *[H]] | 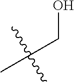 [SMILES: *CO] | hNT-4 | 2 |
| 5a(iii) | -DGKQ- | 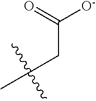 [SMILES: *CC(=O)[O-]] | 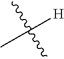 [SMILES: *[H]] | 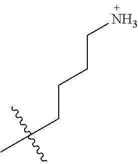 [SMILES: *CCCCN] | 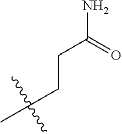 [SMILES: *CCC(N)=O] | hNGF | 3 |
| 5a(iii)m | -DEKQ- | 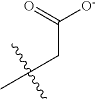 [SMILES: *CC(=O)[O-]] | 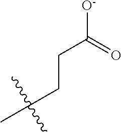 [SMILES: *CCC(=O)[O-]] | 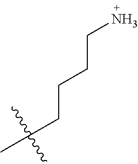 [SMILES: *CCCCN] | 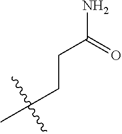 [SMILES: *CCC(N)=O] | mNGF | 3 |
| 5b(iii) | -DSKK- | 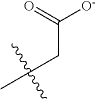 [SMILES: *CC(=O)[O-]] | 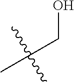 [SMILES: *CO] | 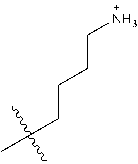 [SMILES: *CCCCN] | 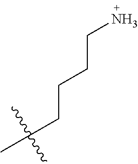 [SMILES: *CCCCN] | hBDNF | 3 |
| 5c(iii) | -ENNK- | 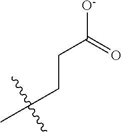 [SMILES: *CCC(=O)[O-]] | 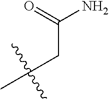 [SMILES: *CC(N)=O] | 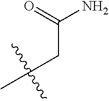 [SMILES: *CC(N)=O] | 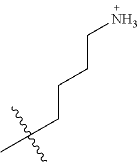 [SMILES: *CCCCN] | hNT-3/ mNT-3 | 3 |
| 5d(iii) | -DAQG- | 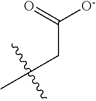 [SMILES: *CC(=O)[O-]] | 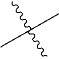 | 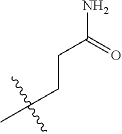 [SMILES: *CCC(N)=O] | 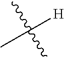 [SMILES: *[H]] | hNT-4 | 3 |

*Note:
a preceding “h” and “m” refer to human and mouse, accordingly

### Characterization of Series 5 Compounds

5a(i)

The purity was found to be 98% by HPLC analysis at 280 and 550 nm detection, retention time 8.662 min. ¹H NMR (400 MHz, 90% H₂O+10% D₂O) δ 8.46 (d, J=7.29 Hz, 1H), 8.41 (d, J=6.45 Hz, 1H), 8.18 (d, J=5.87 Hz, 1H), 8.10 (t, J=6.38 Hz, 1H), 7.98 (d, J=7.91 Hz, 1H), 7.72 (d, J=6.63 Hz, 1H), 7.65 (d, J=9.23 Hz, 2H), 7.57 (s, 3H), 7.45 (s, 1H), 7.37 (s, 1H), 7.22 (d, J=9.25 Hz, 2H), 7.14 (s, 2H), 4.14-4.15 (m, 1H), 4.10-4.12 (m, 1H), 3.91-3.97 (m, 2H), 3.94 (s, 3H), 3.74 (dd, J=13.86, 6.27 Hz, 2H), 3.58-3.60 (m, 1H), 3.55-3.57 (m, 1H), 2.90-2.96 (m, 2H), 2.79-2.86 (m, 2H), 2.04-2.06 (m, 2H), 1.77-1.82 (m, 1H), 1.72-1.96 (m, 1H), 1.59-1.68 (m, 2H), 1.38-1.44 (m, 2H), 1.28-1.36 (m, 1H), 1.08-1.18 (m, 1H), 0.83 (t, J=7.96 Hz, 3H), 0.71 (t, J=7.96 Hz, 3H) High Resolution ESI−: m/z calcd for [C₄₂H₅₃BF₂N₁₀O₁₆S₄]²⁻ 565.1287 found 565.1305.

5b(i)

The purity was found to be 91% by HPLC analysis at 280 and 550 nm detection, retention time 7.438 min. ¹H NMR (400 MHz, 90% H₂O+10% D₂O) δ 8.55 (d, J=7.10 Hz, 1H), 8.45 (d, J=5.76 Hz, 1H), 8.33 (s, 1H), 8.31 (s, 1H), 8.14 (d, J=12.66 Hz, 1H), 7.86 (d, J=7.89 Hz, 2H), 7.65 (d, J=9.37 Hz, 2H), 7.46 (s, 1H), 7.36 (s, 1H), 7.22 (d, J=9.37 Hz, 2H), 7.08 (s, 2H), 4.29-4.33 (m, 1H), 4.07-4.17 (m, 1H), 3.94 (s, 3H), 3.92 (s, 1H), 3.91 (s, 1H), 3.90 (s, 2H), 3.88 (s, 1H), 3.86 (s, 1H), 3.82 (d, J=5.64 Hz, 2H), 3.95 (d, J=5.09 Hz, 1H), 3.71 (d, J=5.13 Hz, 1H), 3.58 (d, J=3.63 Hz, 1H), 3.54 (d, J=5.38 Hz, 1H), 2.74-2.85 (m, 2H), 2.60-2.71 (m, 2H), 2.12-2.21 (m, 2H), 2.04 (s, 3H), 1.99-2.06 (m, 3H) High Resolution ESI−: m/z caled for [C₃₈H₄₄BF₂N₉O₁₇S₅]²⁻ 553.5754 found 553.5773.

5c(i)

The purity was found to be 100% by HPLC analysis at 280 and 550 nm detection, retention time 7.035 min. ¹H NMR (400 MHz, 90% H₂O+10% D₂O) δ 8.45 (d, J=6.91 Hz, 1H), 8.42 (d, J=6.15 Hz, 1H), 8.10 (s, 1H), 8.07 (s, 1H), 8.05 (s, 1H), 7.75 (d, J=7.07 Hz, 1H), 7.62 (d, J=9.41 Hz, 2H), 7.55 (s, 1H), 7.44 (s, 1H), 7.32 (s, 1H), 7.19 (d, J=9.45 Hz, 2H), 7.05-7.12 (m, 4H), 4.15-4.19 (m, 1H), 4.09-4.13 (m, 1H), 3.98 (d, J=5.36 Hz, 2H), 3.93 (s, 3H), 3.88 (d, J=5.36 Hz, 2H), 3.75 (d, J=6.59 Hz, 1H), 3.72 (d, J=6.28 Hz, 1H), 3.62 (d, J=6.43 Hz, 1H), 3.52-3.59 (m, 1H), 3.06 (d, J=6.60 Hz, 2H), 2.90-2.96 (m, 1H), 2.82-2.88 (m, 2H), 2.04 (s, 3H), 1.78-1.83 (m, 1H), 1.71-1.76 (m, 2H), 1.53-1.57 (m, 2H), 1.27-1.35 (m, 1H), 1.07-1.18 (m, 1H), 0.84 (d, J=7.36 Hz, 3H), 0.73 (t, J=16.47 Hz, 3H) High Resolution ESI−: m/z caled for [C₄₂H₅₃BF₂N₁₂O₁₆S₄]²⁻ 579.1318 found 579.1332.

5d(i)

The purity was found to be 91% by HPLC analysis at 280 and 550 nm detection, retention time 6.861 min. ¹H NMR (400 MHz, 90% H₂O+10% D₂O) δ 8.56 (d, J=6.31 Hz, 1H), 8.41 (d, J=6.21 Hz, 1H), 8.08 (d, J=6.56 Hz, 1H), 8.04 (s, 1H), 8.02 (s, 1H), 8.01 (s, 1H), 7.65 (d, J=9.35 Hz, 2H), 7.48 (s, 1H), 7.45 (s, 1H), 7.35 (s, 1H), 7.21 (d, J=9.40 Hz, 2H), 7.09 (s, 2H), 4.31-4.34 (m, 1H), 4.19-4.24 (m, 1H), 4.09-4.10 (m, 2H), 4.05 (d, J=7.33 Hz, 1H), 3.87-3.91 (m, 2H), 3.84 (d, J=5.12, 1H), 3.58-3.75 (m, 2H), 3.45-3.52 (m, 1H), 3.03-3.08 (m, 2H), 2.65-2.83 (m, 2H), 2.06 (s, 3H), 1.72-1.79 (m, 2H), 1.60-1.68 (m, 2H), 1.53-1.59 (m, 2H), 1.22 (s, 1H), 0.82-0.89 (m, 6H) High Resolution ESI−: m/z caled for [C₄₂H₅₃BF₂N₁₂O₁₆S₄]²⁻ 579.1318 found 579.1332.

5a(ii)

The purity was found to be 98% by HPLC analysis at 280 and 550 nm detection, retention time 7.996 min. ¹H NMR (400 MHz, 90% H₂O+10% D₂O) δ 8.49 (d, J=7.30 Hz, 1H), 8.42 (s, 1H), 8.41 (s, 1H), 8.28 (d, J=7.59 Hz, 1H), 8.13, (d, J=7.78 Hz, 1H), 7.85 (d, J=6.82 Hz, 1H), 7.73 (d, J=6.40 Hz, 1H), 7.66 (d, J=9.37 Hz, 2H), 7.52 (s, 1H), 7.49 (s, 1H), 7.33 (s, 1H), 7.22 (d, J=9.36 Hz, 2H), 7.10 (s, 1H), 7.05 (s, 1H), 6.82 (s, 1H), 4.14-4.16 (m, 1H), 4.10-4.12 (m, 1H), 3.97 (s, 1H), 3.96 (s, 1H), 3.94 (s, 1H), 3.75-3.79 (m, 1H), 3.69-3.73 (m, 1H), 3.55-3.60 (m, 1H), 3.48-3.54 (m, 1H), 2.83-2.88 (m, 2H), 2.67-2.73 (m, 2H), 2.20-2.28 (m, 2H), 2.05 (s, 3H), 1.83-1.88 (m, 1H), 1.45-1.53 (m, 1H), 1.16-1.28 (m, 1H), 0.90 (d, J=7.64 Hz, 3H), 0.85 (d, J=8.00 Hz, 3H). High Resolution ESI−: m/z caled for [C₄₁H₅₀BF₂N₁₁O₁₇S₄]²⁻ 572.6161 found 572.6172.

5b(ii)

The purity was found to be 96% by HPLC analysis at 280 and 550 nm detection, retention time 7.619 min. ¹H NMR (400 MHz, 90% H₂O+10% D₂O) δ 8.43 (d, J=6.54 Hz, 1H), 8.38 (d, J=6.83 Hz, 1H), 8.16, (d, J=7.07 Hz, 1H), 8.12, (d, J=7.97 Hz, 1H), 7.88, (d, J=5.96 Hz, 1H), 7.77, (d, J=7.11 Hz, 1H), 7.65 (d, J=9.33 Hz, 2H), 7.52 (s, 1H), 7.47 (s, 1H), 7.33, (s, 1H), 7.21 (d, J=9.44 Hz, 2H), 7.10 (s, 1H), 4.18-4.23 (m, 1H), 4.02 (s, 1H), 3.97 (s, 1H), 3.94 (s, 3H), 3.88 (s, 2H), 3.86 (s, 2H), 3.67-3.69 (m, 1H), 3.63-3.66 (m, 2H), 3.58-3.60 (m, 2H), 3.55-3.56 (m, 1H), 2.82-2.85 (m, 2H), 2.15 (m, 1H), 2.06 (s, 3H), 1.61-1.70 (m, 2H), 1.44-1.54 (m, 2H), 1.22-1.26 (m, 2H), 0.96 (t, J=8.63 Hz, 6H). High Resolution ESI−: m/z caled for [C₄₀H₅₁BF₂N₁₀O₁₅S₄]²⁻ 544.1234 found 544.1248.

5c(ii)

The purity was found to be 96% by HPLC analysis at 280 and 550 nm detection, retention time 7.236 min. ¹H NMR (400 MHz, 90% H₂O+10% D₂O) δ 8.51 (t, J=5.82 Hz, 1H), 8.48 (s, 1H), 8.46 (s, 1H), 8.36 (d, J=7.21 Hz, 1H), 8.29 (d, J=7.84 Hz, 1H), 8.10 (d, J=6.76 Hz, 1H), 7.65 (d, J=10.19 Hz, 2H), 7.50 (s, 1H), 7.46 (s, 1H), 7.37 (s, 1H), 7.21 (d, J=8.83 Hz, 2H), 4.27-4.41 (m, 1H), 4.04-4.05 (m, 1H), 4.00 (s, 1H), 3.98 (s, 1H), 3.97 (s, 1H), 3.93 (s, 2H), 3.92 (s, 1H), 3.87-3.89 (m, 2H), 3.63-3.68 (m, 2H), 3.54-3.60 (m, 2H), 2.87-2.92 (m, 2H), 2.72-2.78 (m, 2H), 2.00-2.05 (m, 1H), 2.03 (s, 3H), 1.21 (d, J=6.44 Hz, 3H). High Resolution ESI−: m/z caled for [C₄₀H₄₈BF₂N₁₁O₁₈S₄]²⁻ 573.6056 found 573.6074.

5c(ii)m

The purity was found to be 97% by HPLC analysis at 280 and 550 nm detection, retention time 8.788 min. ¹H NMR (400 MHz, 90% H₂O+10% D₂O) δ 8.50 (s, 1H), 8.47 (s, 1H), 8.07 (d, J=7.70 Hz, 1H), 7.91 (d, J=6.85 Hz, 1H), 7.65 (d, J=9.71 Hz, 2H), 7.47 (s, 1H), 7.40 (s, 1H), 7.35 (s, 1H), 7.22 (d, J=9.74 Hz, 2H), 7.10 (s, 1H), 4.28-4.33 (m, 1H), 4.13-4.18 (m, 1H) 3.94 (s, 3H), 3.88 (d, J=5.98 Hz, 2H), 3.85 (d, J=5.77 Hz, 1H), 3.81 (d, J=4.41 Hz, 1H), 3.78 (d, J=4.77 Hz, 2H), 3.63 (d, J=6.45 Hz, 2H), 3.45 (dd, J=15.17, 11.09 Hz, 1H), 2.45-2.50 (m, 2H), 2.31 (t, J=7.88 Hz, 2H), 2.08-2.15 (m, 1H), 2.16 (s, 3H), 1.94-2.00 (m, 1H), 1.23 (d, J=6.37 Hz, 3H). High Resolution ESI−: m/z caled for [C₃₇H₄₃BF₂N₁₀O₁₇S₄]²⁻ 538.0870 found 538.0884.

5d(ii)

The purity was found to be 92% by HPLC analysis at 280 and 550 nm detection, retention time 6.624 min. ¹H NMR (400 MHz, 90% H₂O+10% D₂O) δ 8.65 (d, J=3.91 Hz, 1H), 8.36 (d, J=6.09 Hz, 1H), 8.32 (d, J=7.58 Hz, 1H), 8.26 (s, 1H), 8.23 (d, J=6.73 Hz, 1H), 7.67 (d, J=10.03 Hz, 2H), 7.44 (s, 1H), 7.41 (s, 1H), 7.22 (d, J=10.03 Hz, 2H), 7.07 (s, 1H), 4.24-4.28 (m, 1H), 4.08-4.14 (m, 1H), 4.05 (s, 1H), 3.98 (d, J=5.06, 2H), 3.92 (d, J=5.40 Hz, 2H), 3.81-3.82 (m, 1H), 3.74-3.78 (m, 2H), 3.66-3.70 (m, 2H), 3.49-3.55 (m, 2H), 1.98 (s, 3H), 1.41 (d, J=7.76 Hz, 3H). High Resolution ESI−: m/z caled for [C₃₄H₃₈BF₂N₉O₁₅S₄]²⁻ 494.5710 found 494.5721.

5a(iii)

The purity was found to be 100% by HPLC analysis at 280 and 550 nm detection, retention time 6.541 min. ¹H NMR (400 MHz, 90% H₂O+10% D₂O) δ 9.08 (d, J=6.30 Hz, 1H), 8.27 (d, J=6.15 Hz, 1H), 8.13 (t, J=5.95 Hz, 1H), 7.86 (d, J=7.38 Hz, 1H), 7.71 (d, J=5.49 Hz, 1H), 7.65 (d, J=9.44 Hz, 2H), 7.46 (s, 1H), 7.39 (s, 1H), 7.34 (s, 3H), 7.21 (d, J=9.45 Hz, 2H), 7.10 (s, 2H), 4.22-4.30 (m, 1H), 4.13-4.19 (m, 1H), 3.94 (s, 3H), 3.77-3.80 (m, 1H), 3.76-3.79 (m, 1H), 3.68-3.72 (m, 1H), 3.67-3.70 (m, 1H), 3.30 (d, J=11.86 Hz, 1H), 3.27 (d, J=11.94 Hz, 1H), 2.91-2.96 (m, 2H), 2.81-2.88 (m, 1H), 2.15-2.23 (m, 2H), 2.07-2.12 (m, 2H), 2.05 (s, 3H), 2.02-2.07 (m, 2H), 1.78-1.89 (m, 2H), 1.56-1.64 (m, 2H), 1.39-1.47 (m, 2H) High Resolution ESI−: m/z caled for [C₄₁H₅₀BF₂N₁₁O₁₇S₄]²⁻ 572.6160 found 572.6175.

5a(iii)m

The purity was found to be 97% by HPLC analysis at 280 and 550 nm detection, retention time 7.036 min. ¹H NMR (400 MHz, 90% H₂O+10% D₂O) δ 8.58 (d, J=5.00 Hz, 1H), 8.31 (d, J=7.20 Hz, 1H), 8.27 (d, J=7.31 Hz, 1H), 8.10 (d, J=7.36 Hz, 1H), 8.06 (d, J=7.29 Hz, 1H), 9.91 (d, J=7.01 Hz, 1H), 7.65 (d, J=9.79 Hz, 2H), 7.52 (s, 3H), 7.46 (S, 1H), 7.39 (S, 1H), 7.21 (d, J=10.01 Hz, 2H), 7.07 (S, 2H), 4.26-4.33 (m, 1H), 4.18-4.24 (m, 1H), 3.94 (S, 3H), 3.90-3.92 (m, 1H), 3.85-3.86 (m, 1H), 3.76-3.83 (m, 2H), 3.65-3.72 (m, 2H), 3.62 (d, J=4.72 Hz, 1H), 3.59 (d, J=4.69 Hz, 1H), 2.95-3.02 (m, 2H), 2.83-2.93 (m, 2H), 3.62 (d, J=4.72 Hz, 1H), 3.59 (d, J=4.69 Hz, 1H), 2.95-3.02 (m, 2H), 2.83-2.93 (m, 2H), 2.43 (t, J=16.13 Hz, 2H), 2.25 (t, J=19.46 Hz, 2H), 2.18 (t, J=19.46, 2H), 2.01 (s, 3H), 9.89-9.99 (m, 1H), 1.81-1.88 (m, 2H), 1.60-1.66 (m, 2H), 1.35-1.42 (m, 2H) High Resolution ESI−: m/z caled for [C₄₄H₅₄BF₂N₁₁O₁₉S₄]²⁻ 608.6265 found 608.6286.

5b(iii)

The purity was found to be 99% by HPLC analysis at 280 and 550 nm detection, retention time 8.897 min. ¹H NMR (400 MHz, 90% H₂O+10% D₂O) δ 8.88 (d, J=6.79 Hz, 1H), 8.39 (d, J=6.02 Hz, 1H), 7.91 (d, J=5.40 Hz, 1H), 7.72 (d, J=7.18 Hz, 1H), 7.64 (d, J=9.27 Hz, 2H), 7.58 (t, J=17.35 Hz, 1H), 7.45 (s, 1H), 7.40 (s, 1H), 7.31 (s, 1H), 7.21 (d, J=9.32 Hz, 2H), 7.10 (s, 2H), 4.18 (d, J=4.05 Hz, 1H), 4.15 (d, J=3.52 Hz, 1H), 4.06 (s, 1H), 4.04 (s, 1H), 3.94 (s, 3H), 3.75 (t, J=10.59 Hz, 2H), 3.37-3.40 (m, 1H), 3.40-3.37 (m, 1H), 3.05 (d, J=7.31 Hz, 1H), 3.01 (s, 1H), 2.97 (s, 2H), 2.85 (s, 2H), 2.80 (d, J=6.74 Hz, 1H), 2.75 (d, J=6.60 Hz, 1H), 2.03 (s, 3H), 1.98-2.04 (m, 2H), 1.81-1.87 (m, 2H), 1.70-1.81 (m, 2H), 1.64-1.66 (m, 2H), 1.59-1.62 (m, 2H), 1.48-1.55 (m, 2H), 1.36-1.46 (m, 2H), 1.26-1.28 (m, 2H), 1.22-1.24 (m, 2H) High Resolution ESI−: m/z caled for [C₄₃H₅₆BF₂N₁₁O₁₇S₄]²⁻ 587.6394 found 587.6413.

5c(iii)

The purity was found to be 98% by HPLC analysis at 280 and 550 nm detection, retention time 6.783 min. ¹H NMR (400 MHz, 90% H₂O+10% D₂O) δ 8.77 (d, J=6.29 Hz, 1H), 8.37 (d, J=6.86 Hz, 1H), 8.30 (d, J=7.67 Hz, 1H), 8.11 (d, J=7.52 Hz, 1H), 7.97-8.01 (m, 1H), 7.93 (d, J=6.00 Hz, 1H), 7.65 (d, J=9.48 Hz, 2H), 7.51 (s, 1H), 7.47 (s, 1H), 7.38 (s, 1H), 3.36 (s, 3H), 7.22 (d, J=9.55 Hz, 2H), 7.10 (s, 1H), 6.77 (s, 2H), 4.20-4.25 (m, 1H), 4.13-4.18 (m, 1H), 3.94 (s, 3H), 3.96 (s, 1H), 3.91 (s, 1H), 3.71-3.76 (m, 1H), 3.61-3.66 (m, 1H), 3.42 (d, J=11.84 Hz, 1H), 3.38 (d, J=11.53 Hz, 1H), 2.86-2.70 (m, 2H), 2.74-2.85 (m, 2H), 2.51-2.52 (m, 2H), 2.45-2.49 (m, 2H), 2.08-2.18 (m, 2H), 2.06 (s, 3H), 1.68-1.78 (m, 2H), 1.48-1.56 (m, 2H), 1.41-1.46 (m, 2H), 1.22-1.28 (m, 2H) High Resolution ESI−: m/z caled for [C₄₃H₅₃BF₂N₁₂O₁₈S₄]²⁻ 601.1267 found 601.1285.

5d(iii)

The purity was found to be 99% by HPLC analysis at 280 and 550 nm detection, retention time 7.078 min. ¹H NMR (400 MHz, 90% H₂O+10% D₂O) δ 8.55 (t, J=7.58 Hz, 1H), 8.30-8.32 (m, 1H), 8.26-8.28 (m, 1H), 8.09-8.11 (m, 1H), 8.00-8.01 (m, 1H), 7.89-7.99 (m, 1H), 7.65 (d, J=9.33 Hz, 2H), 7.46 (s, 1H), 7.21 (d, J=9.33 Hz, 2H), 7.10 (s, 1H), 7.05 (s, 1H), 6.92 (s, 1H), 4.21-4.25 (m, 1H), 4.04-4.06 (m, 1H), 3.97-4.00 (m, 1H), 3.94 (s, 3H), 3.88-3.91 (m, 2H), 3.84-3.85 (m, 1H), 3.78-3.80 (m, 1H), 3.75-3.76 (m, 1H), 3.57-3.61 (m, 1H), 3.52-3.55 (m, 1H), 2.86-2.34 (m, 2H), 2.46 (t, J=7.83 Hz, 1H), 2.33 (t, J=7.85 Hz, 1H), 2.07-2.14 (m, 2H), 2.02 (s, 1H), 1.42 (dd, J=7.13, 3.80 Hz, 3H). High Resolution ESI−: m/z caled for [C₃₈H₄₃BF₂N₁₀O₁₇S₄]²⁻ 544.0870 found 544.0884.

### Biological Assay Methods

### Cytotoxicity Assays

Cells were plated in 96-well plates at a density of 2000 cells/well and let adhere for 24 hours. Compounds were added to a maximum concentration of 100 μM to determine if they have any cytotoxic effects. Compounds were incubated with cells for 48-72 hours, after which cell viability was determined via an alamarBlue assay and normalized to cells grown in complete media to 100% survival. Gambogic amide is used as a cytotoxic control.

No significant cytotoxic effects were seen by any tested compounds of Series 1-5 in HeLa-TrkA cells up to 100 μM. No significant cytotoxic effects were seen by any tested compounds of Series 5 in HeLa293-TrkB cells up to 100 μM. No significant cytotoxic effects were seen by any tested compounds of Series 1-5 in NIH3T3-TrkC cells up to 100 μM.

### Cell Survival Compound Screen

Cells were seeded at a density of 2000 cells per well. Cells were incubated in complete media for 24 hours. The media was aspirated, the cells washed twice with Dulbecco's Phosphate Buffered Saline, and the media was replaced with serum-free media to induce apoptosis unless otherwise halted. Compound was added to cells (50 μM compound to HeLa-TrkA and NIH3T3-TrkC cells, 0.4 μM for HEK293-TrkB) alone (true agonism) or in the presence of suboptimal neurotrophin (˜25 to 30% survival, 0.2 nM NGF, 0.6 nM BDNF, or 0.2 nM NT3 for TrkA, B, or C expressing cells respectively). Cell survival was measured via alamarBlue assay after 48-72 hours to determine cell viability. Data was normalized to DMSO treatment (0%) and optimal neurotrophin (100%, 2.0 nM NGF, 1.0 nM BDNF, 2.0 nM NT3 for TrkA, B, or C expressing cells, respectively).

### Cell Survival of HeLa-TrkA Cells

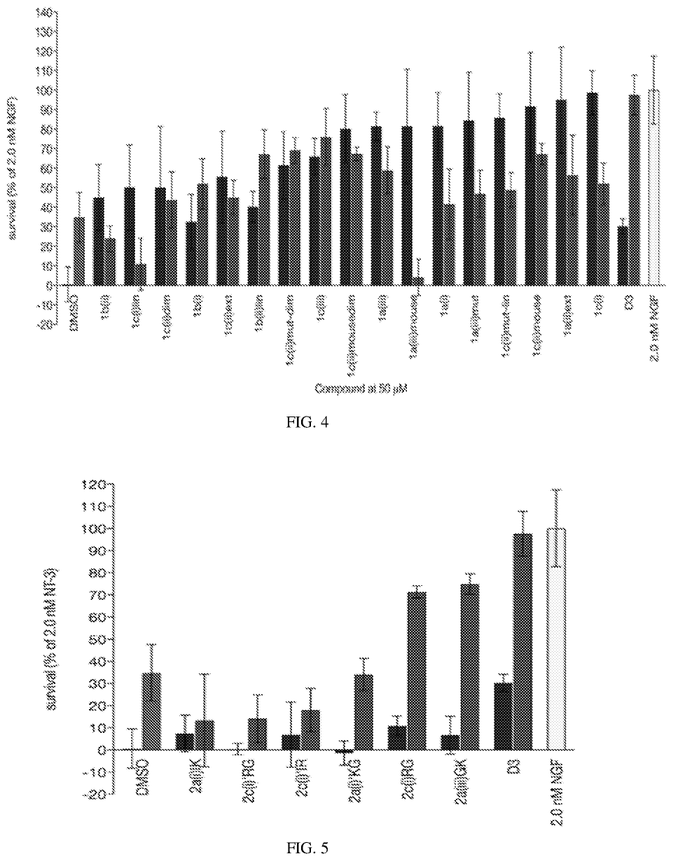

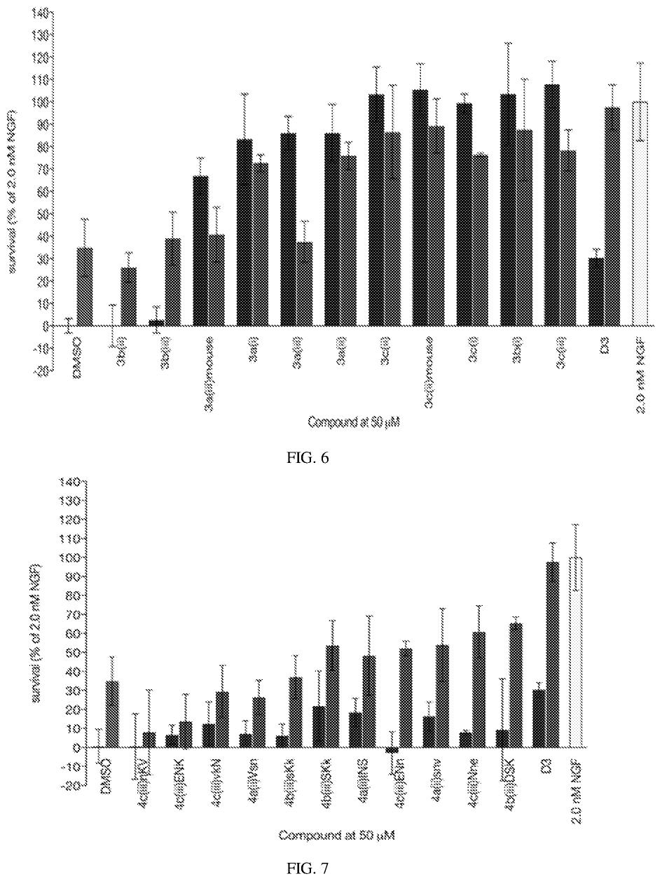

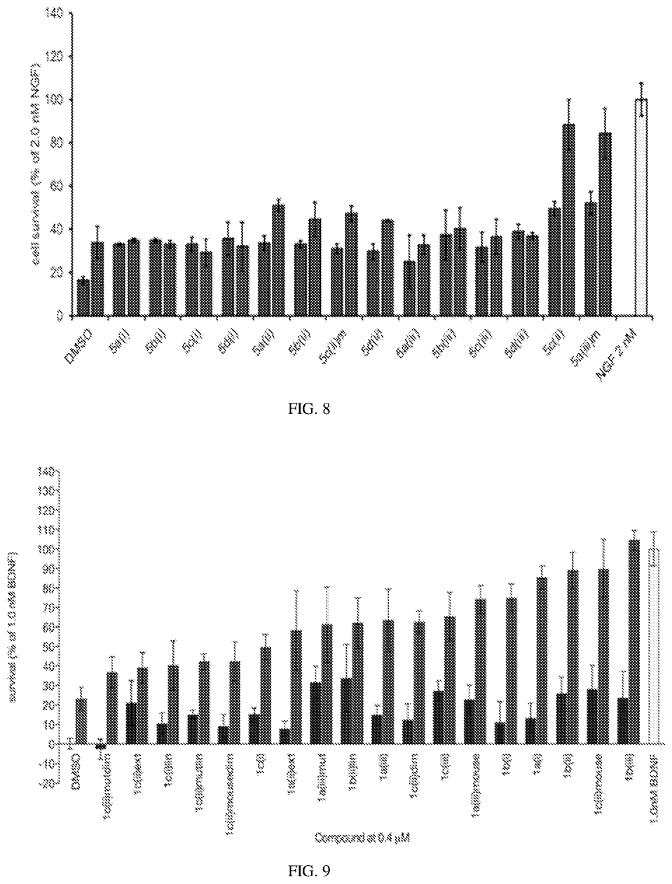

HeLa-TrkA cells were treated with 50 μM compound with or without suboptimal (0.2 nM) levels of NGF and cell survival was analyzed after 48-72 hours by the alamarBlue assay. Data was normalized to DMSO (0%) and 2.0 nM NGF (100%). Data is represented as the average of 3-6 points +/−standard deviation from the mean. Results for Series 1-5 compounds can be found in FIGS. 4-8, respectively.

### Cell Survival of HEK293-TrkB Cells

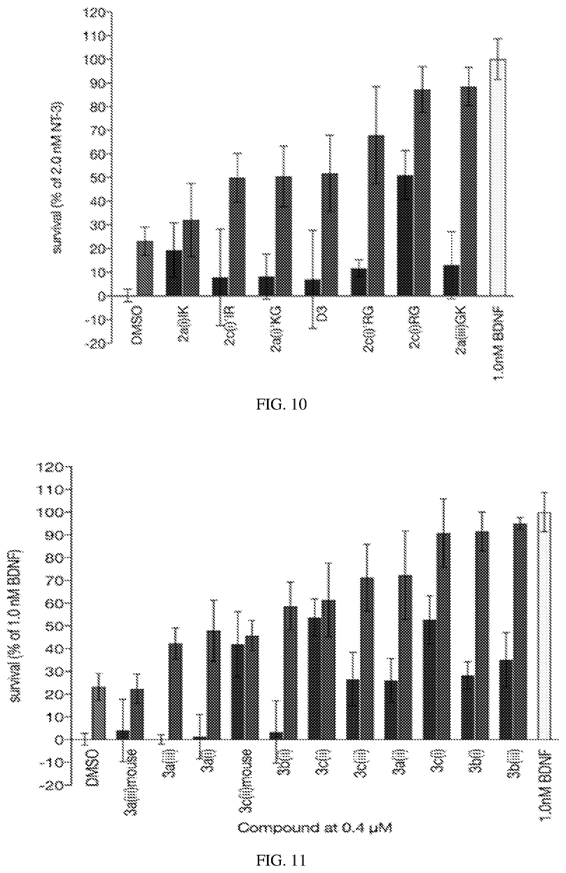

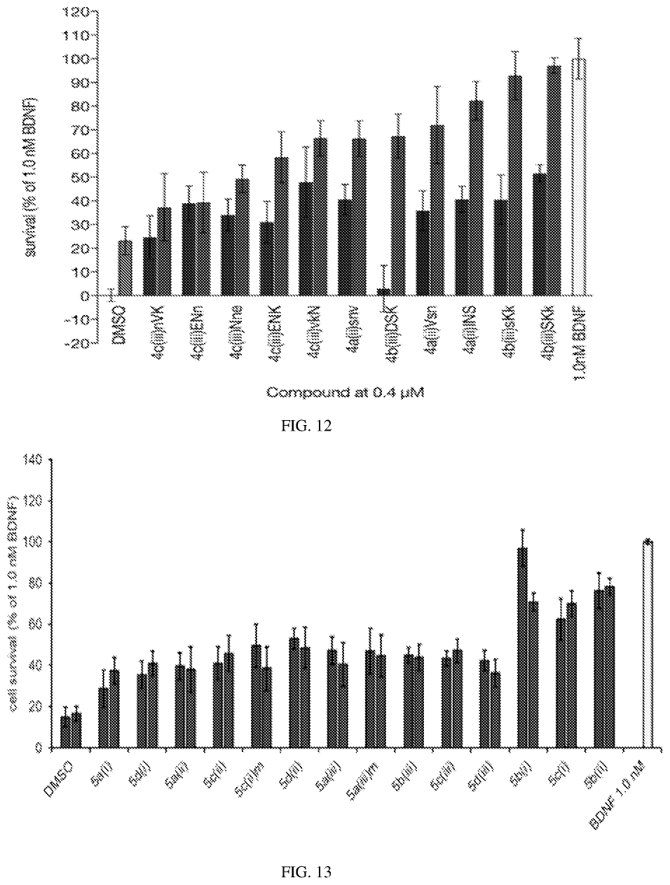

HEK293-TrkB cells were treated with 0.4 μM compound with or without suboptimal (0.6 nM) levels of BDNF and cell survival was analyzed after 48-72 hours by the alamarBlue assay. Data was normalized to DMSO (0%) and 1.0 nM BDNF (100%). Data is represented as the average of 3-6 points +/−standard deviation from the mean. Results for Series 1-5 compounds can be found in FIGS. 9-13, respectively.

### Cell Survival of NIH3T3-TrkC Cells

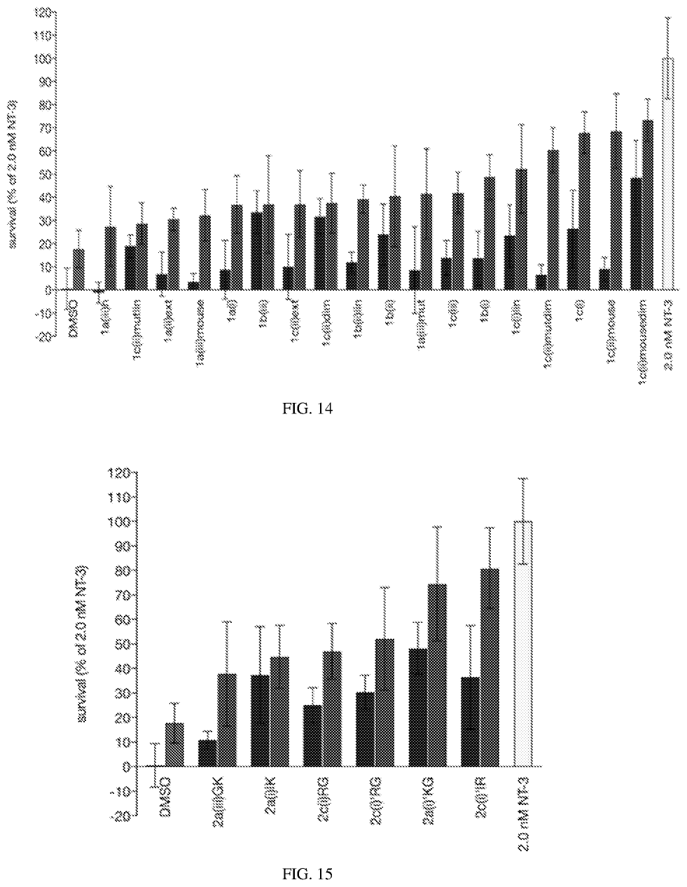

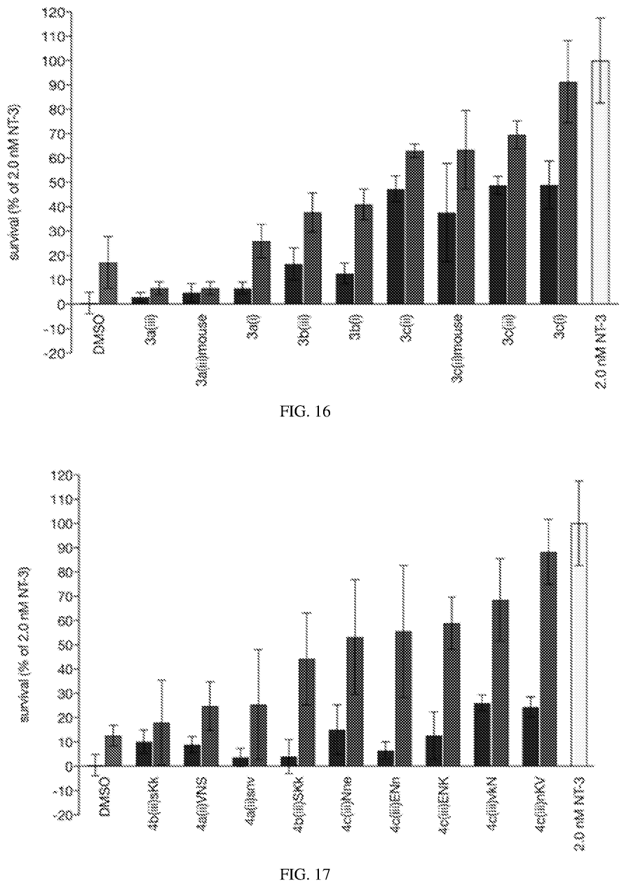

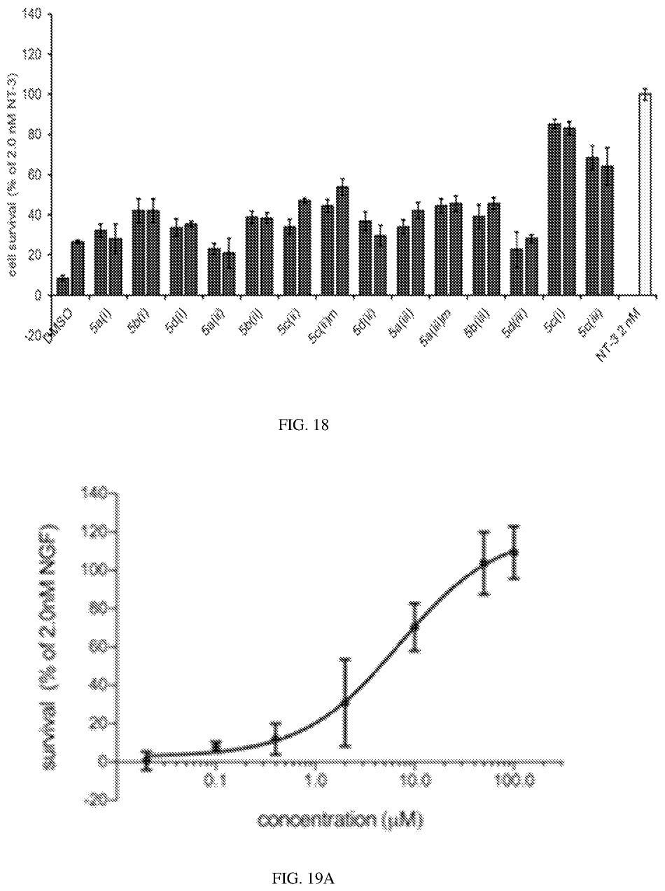

NIH3T3-TrkC cells were treated with 0.4 μM compound with or without suboptimal (0.2 nM) levels of NT3 and cell survival was analyzed after 48-72 hours by the alamarBlue assay. Data was normalized to DMSO (0%) and 2.0 nM NT3 (100%). Data is represented as the average of 3-6 points +/−standard deviation from the mean. Results for Series 1-5 compounds can be found in FIGS. 14-18, respectively

### Cell Survival Dose Response

### General Dose Response Procedure

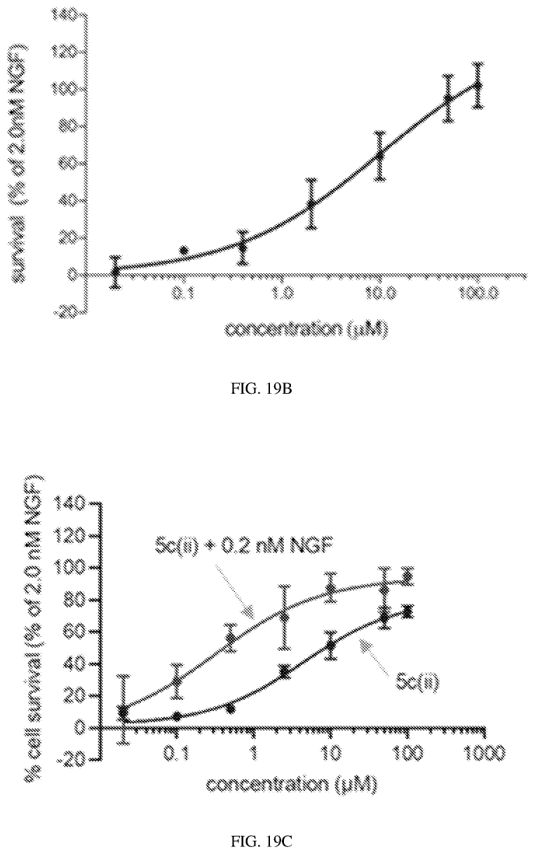

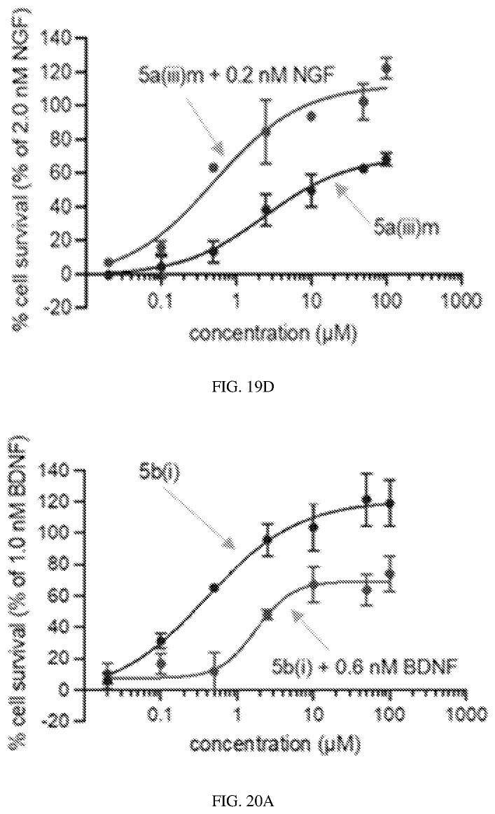

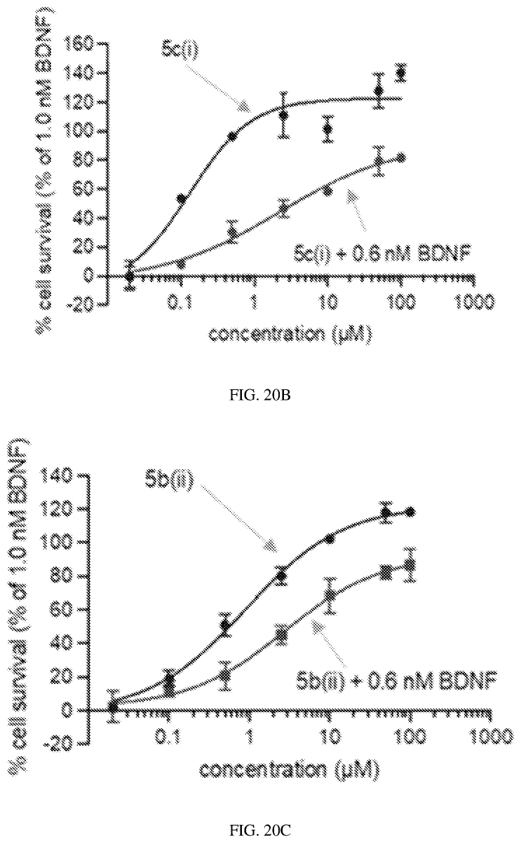

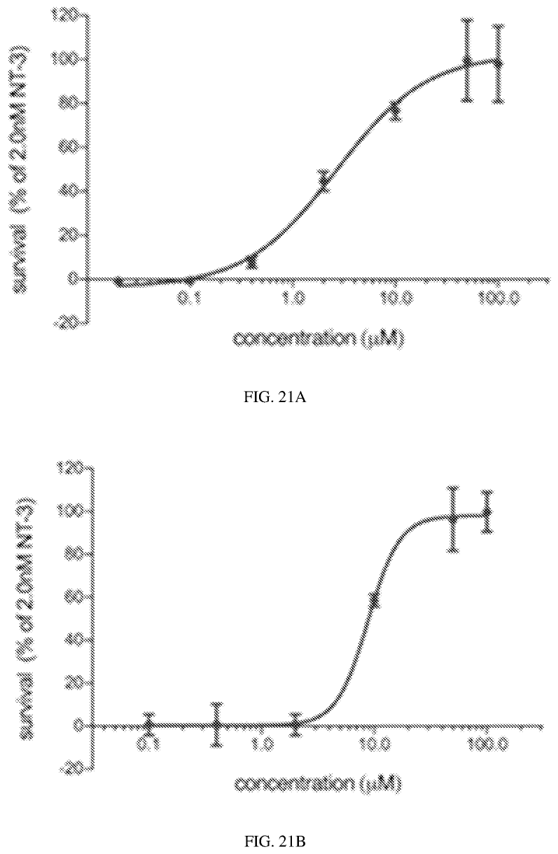

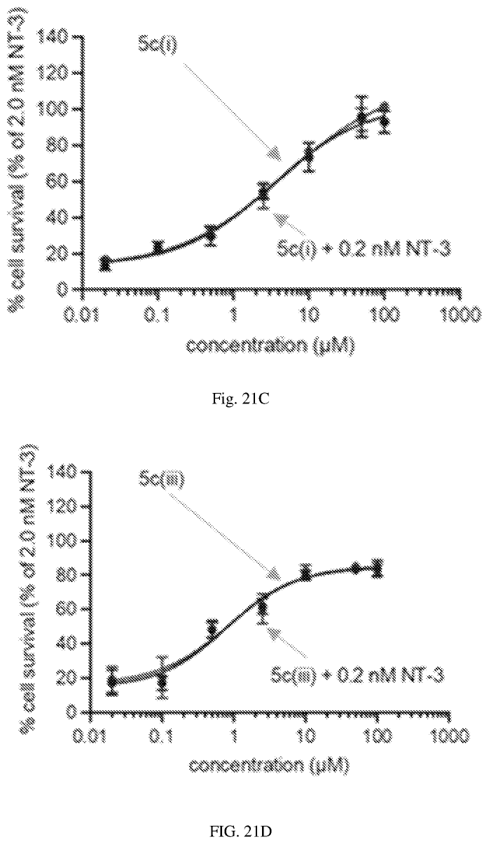

The most promising compounds from the screen were selected for each cell line. Cells were treated with a serial dilution of the compound in serum free media with or without suboptimal neurotrophin and incubated for 48-72 hours, after which an alamarBlue assay was done to determine cell viability. Cell viability was normalized to DMSO (0%) and optimal neurotrophin (100%), depending on cell type. EC₅₀ was calculated in Graphpad Prism using the dose response “[agonist] vs response—Variable slope” function. Data is presented as the mean +/−standard deviation of an experiment with 3-6 replicates. Results for selected compounds with HeLa-TrkA cells can be found in FIG. 19A-19D. Results for selected compounds with HEK293-TrkB cells can be found in FIG. 20A-20C. Results for selected compounds with NIH3T3-TrkC cells can be found in FIG. 21A-21D.

### HeLa-TrkA Cell Survival Dose Response

TABLE 6
Results of the HeLa-TrkA Cell Survival Dose
Response determining agonism and EC₅₀.

| Compound Name | Partial or True Agonism | EC₅₀ (μM) |
|---|---|---|
| 1a(ii)ext | true | 7.07 |
| 3a(ii) | true | 10.04 |
| 5c(ii) | true | 4.5 |
| 5c(ii) + 0.2 nM NGF | partial | 0.3 |
| 5a(iii)m | true | 2.4 |
| 5a(iii)m + 0.2 nM NGF | partial | 0.5 |

### HEK293-TrkB Cell Survival Dose Response

TABLE 7
Results of the HEK293-TrkB Cell Survival Dose
Response determining agonism and EC₅₀.

| Compound Name | Partial or True Agonism | EC₅₀ (μM) |
|---|---|---|
| 5b(i) | true | 0.4 |
| 5b(i) + 0.6 nM BDNF | partial | 1.8 |
| 5c(i) | true | 0.1 |
| 5c(i) + 0.6 nM BDNF | partial | 2.2 |
| 5b(ii) | true | 0.9 |
| 5b(ii) + 0.6 nM BDNF | partial | 2.8 |

NIH3T3-TrkC Cell Survival Dose Response

TABLE 8
Results of the NIH3T3-TrkC Cell Survival Dose
Response determining agonism and EC₅₀.

| Compound Name | Partial or True Agonism | EC₅₀ (μM) |
|---|---|---|
| 3c(i) + 0.2 nM NT3 | partial | 2.6 |
| 4c(iii) + 0.2 nM NT3 | partial | 8.6 |
| 5c(i) | true | 3.3 |
| 5c(i) + 0.2 nM NT3 | partial | 4.9 |
| 5c(iii) | true | 0.8 |
| 5c(iii) + 0.2 nM NT3 | partial | 0.9 |

### K_{d} Determination of Fluorescent Compounds to Cell Surface

### General Procedure for K_{d} Determination

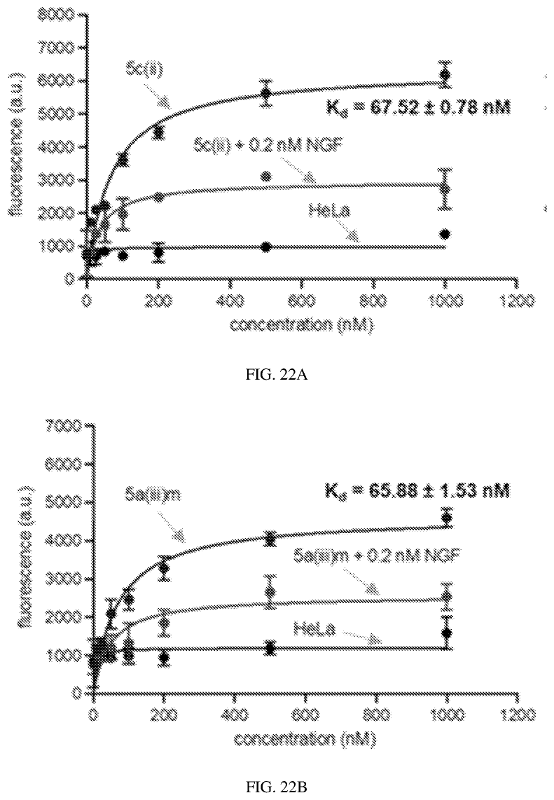

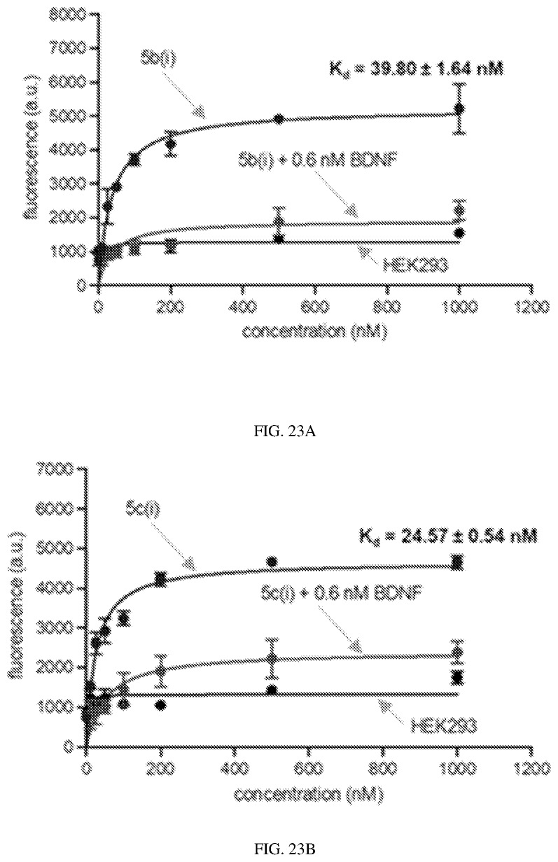

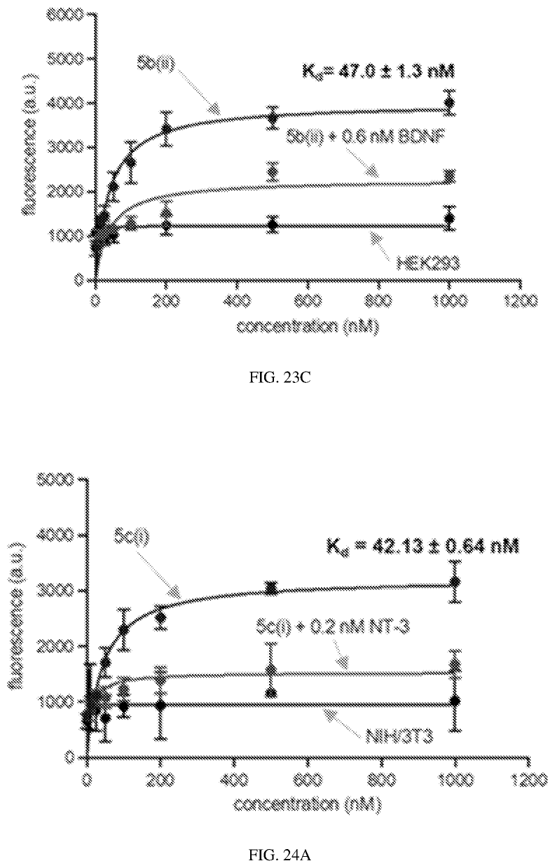

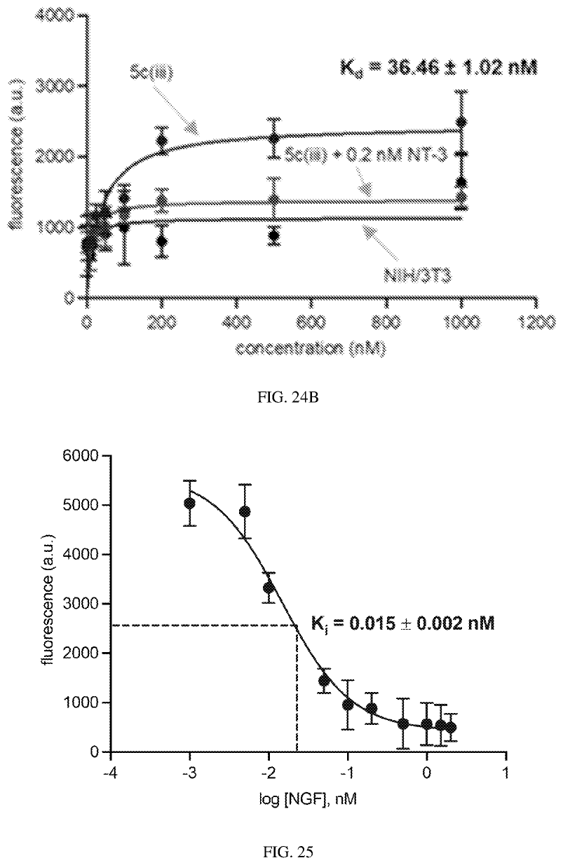

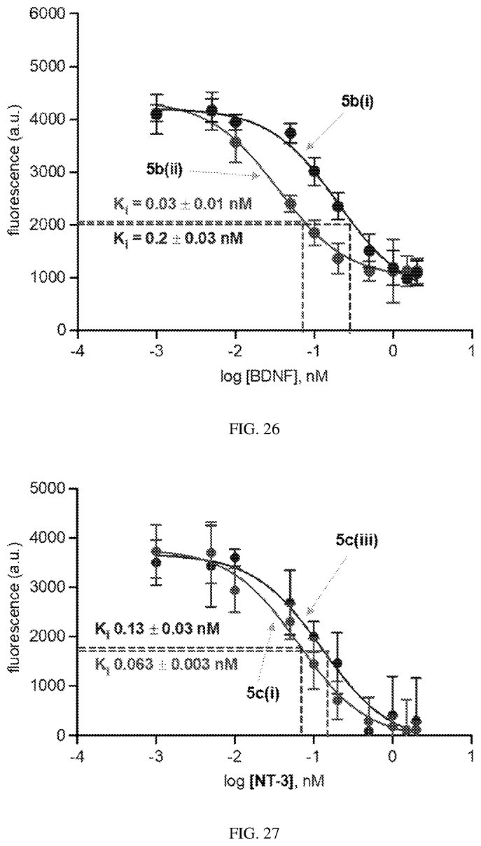

Transfected cell lines that are Trk positive cells (TrkA-HeLa, TrkB-HEK293, and TrkC-NIH/3T3) and non-transfected cell lines that are Trk negative cells (HeLa, HEK293, and NIH/3T3) were seeded approximately 2×10³ cells/well in a 96-well plate and incubated for 24 h. The cells were treated with concentrations serial 0, 10, 25, 50, 100, 200, 500, and 1000 nM of the compounds in serum-free media (SFM) with and without 0.2 nM of NGF (TrkA-HeLa and HeLa), 0.6 nM of BDNF (TrkB-HEK293 and HEK293), and 0.2 nM of NT-3 (TrkC-NIH/3T3 and NIH/3T3) for 2.5 h. After that, the cells were washed with PBS once to remove unbound fluorescence and dissolved in 1% (w/v) aqueous sodium dodecyl sulfate. Cell-associated fluorescence was then determined by measuring the emission of the resulting solution upon λₑₓ (540/25 nm) and λₑₘ (620/40 nm) using Agilent BioTek Synergy H4 Hybrid Microplate Reader. K_{d} and Kᵢ were calculated by GraphPad Prism9 using Binding saturation (One site—Specific binding). Results for K_{d} of selected compounds with HeLa-TrkA cells can be found in FIG. 22A-22B. Results for K_{d} of selected compounds with HEK293-TrkB cells can be found in FIG. 23A-23C. Results for K_{d} of selected compounds with NIH3T3-TrkC cells can be found in FIG. 24A-24B. Results for Kᵢ of selected compounds with HeLa-TrkA cells can be found in FIG. 25. Results for Kᵢ of selected compounds with HEK293-TrkB cells can be found in FIG. 26. Results for Kᵢ of selected compounds with NIH3T3-TrkC cells can be found in FIG. 27.

1. A compound of the formula 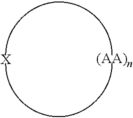
wherein
(AA)ₙ is an amino acid sequence, wherein each AA is an independently selected amino acid, provided that at least one amino acid in the (AA)ₙ sequence is selected from the group consisting of L-arginine (R), D-arginine (r), L-aspartic acid (D), D-aspartic acid (d), L-glutamic acid (E), D-glutamic acid (e), L-lysine (K), D-lysine (k), L-glutamine (Q), D-glutamine (q), L-serine (S), D-serine (s), L-cysteine (C), D-cysteine (c), L-asparagine (N), and D-asparagine (n);
X is a linking group comprising one or more of a heterocycloalkylene portion, a cycloalkylene portion, a divalent triazole portion, or a divalent dye portion; and
n is 3, 4, 5, 6, 7, or 8.

2. The compound of claim 1, wherein one or more amino acids in the amino acid sequence is a naturally occurring amino acid selected from the group consisting of L-histidine (H), L-threonine (T), L-glycine (G), L-proline (P), L-alanine (A), L-valine (V), L-isoleucine (I), L-leucine (L), L-methionine (M), L-phenylalanine (F), L-tyrosine (Y), and L-tryptophan (W).

3. The compound of claim 1, wherein one or more amino acids in the amino acid sequence is a naturally occurring amino acid in the D-configuration selected from the group consisting of D-histidine (h), D-threonine (t), D-glycine (g), D-proline (p), D-alanine (a), D-valine (v), D-isoleucine (i), D-leucine (l), D-methionine (m), D-phenylalanine (f), D-tyrosine (y), and D-tryptophan (w).

4. The compound of claim 1, wherein one or more amino acids in the amino acid sequence is an unnatural amino acid selected from the group consisting of selenocysteine, citrulline (Cit), hydroxyproline (Hyp), norleucine (Nle), ornithine (Orn), naphtylalanine (Nal), methionine sulfoxide, methionine sulfone, beta-alanine, α-aminobutyric acid, γ-aminobutyric acid, diaminobutyric acid, δ-aminolevulinic acid, 4-amino-benzoic acid, hydroxyproline, and carboxyglutamic acid.

5. The compound of claim 1, wherein the amino acid sequence comprises a mimetic of loop-1, loop-2, or loop-3 of a neurotrophin.

6. The compound of claim 1, wherein the neurotrophin is nerve growth factor, brain-derived neurotrophic factor, neurotrophin-3, or neurotrophin-4.

7. (canceled)

8. The compound of claim 1, wherein at least two amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine.

9. The compound of claim 1, wherein at least three amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine.

10. The compound of claim 1, wherein at least four amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine.

11. The compound of claim 1, wherein at least five amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine.

12. The compound of claim 1, wherein at least six amino acid in the (AA)ₙ sequence are independently selected from the group consisting of L-arginine, D-arginine, L-aspartic acid, D-aspartic acid, L-glutamic acid, D-glutamic acid, L-lysine, D-lysine, L-glutamine, D-glutamine, L-serine, D-serine, L-cysteine, D-cysteine, L-asparagine, and D-asparagine.

13.-18. (canceled)

19. The compound of claim 1, wherein the amino acid sequence comprises a sequence selected from the group consisting of -INS-, -snv-, -Vsn-, -DSK-, -SKk-, -sKk-, -Kks-, -ENK-, -nKV-, -vKN-, -Nne-, -ENn-, -DIKG-, -INNS-, -DGKQ-, -DEKQ-, -DMSG-, -VSKG-, -DSKK-, -DIRG-, -TQNS-, -TGNS-, -ENNK-, -DIKGK-, -NINNSVF-, -DGKQA-, -DEKQA-, -DMSGG-, -SKGQ-, -DSKKR-, -DIRGH-, -TQNSP-, -KTQNSPV-, -TQNSG-, -TGNSP-, and -ENNKLV-.

20. The compound of claim 1 wherein X comprises a heterocycloalkylene portion.

21. The compound of claim 20, wherein X is of the formula 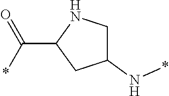 [SMILES: *NC1CNC(C(*)=O)C1]
wherein each * represents a point of covalent attachment to the rest of the compound.

22. The compound of claim 21, wherein X further comprises a dye molecule covalently attached thereto.

23. The compound of claim 22, wherein X has the formula 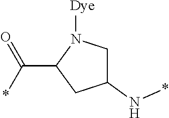
wherein each * represents a point of covalent attachment to the rest of the compound, and dye is a fluorescent dye molecule.

24. (canceled)

25. The compound of claim 1, wherein X comprises a divalent triazole portion.

26. The compound of claim 25, wherein X comprises a divalent triazole of the formula 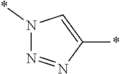 [SMILES: *c1cn(*)nn1]
wherein each * represents a point of covalent attachment to the rest of the compound.

27. The compound of claim 26, wherein X comprises the structure 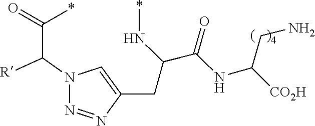
wherein each * represents a point of covalent attachment to the rest of the compound, and R′ is a side chain of the amino acid T, V, M, I, K, or S.

28.-37. (canceled)

38. A method of treating disease in which cell survival is mediated by one or more of TrkA, TrkB, or TrkC, comprising administering to a subject a compound of claim 1.

39. (canceled)
